(19) United States
(12) Patent Application Publication
(10) Pub. No.: US 2021/0386780 A1
(43) Pub. Date: Dec. 16, 2021

(54) METHODS FOR TREATING CANCER WITH DOUBLE STRANDED RNA SENSOR ACTIVATORS AND ADOPTIVE CELL THERAPY

(71) Applicants: The Regents of the University of California, Oakland, CA (US); HIGHLIGHT THERAPEUTICS S.L., Paterna (ES)

(72) Inventors: Anusha KALBASI, Los Angeles, CA (US); Antoni RIBAS, Los Angeles, CA (US); Marisol QUINTERO, Paterna (ES)

(21) Appl. No.: 17/281,551

(22) PCT Filed: Sep. 30, 2019
(86) PCT No.: PCT/US2019/053832

§ 371 Date: Mar. 30, 2021

Related U.S. Application Data
(60) Provisional application No. 62/739,783, filed on Oct. 1, 2018.

(51) Int. Cl. A61K 35/17; A61K 31/713; A61K 47/59; A61P 35/00

(52) U.S. Cl. CPC A61K 35/17; A61P 35/00; A61K 47/59; A61K 31/713

(57) ABSTRACT

Disclosed herein are improved methods of treating cancer in a subject by administering Adoptive Cell Therapy, in particular in those subjects affected by a cancer that presents a loss of function, mutation, or other disruption in an immune pathway. The loss of function mutation or disruption can be in IFNAR1, JAK2, or B2M. The methods include the intratumoral administration of nanoplexed poly(TC) formulations. These methods are further useful for a variety of therapeutic methods and uses relating to the administration of an immune checkpoint therapy such as anti-PD 1 or anti-PDL1 for the prevention of, and/or against the occurrence of cancer, particularly solid cancer.

### CROSS-REFERENCE TO RELATED APPLICATIONS

This application claims priority to and the benefit of U.S. Provisional Application No. 62/739,783, filed Oct. 1, 2018, the contents of which is incorporated into the present application by reference.

### FIELD OF INVENTION

The present invention relates to method for improving adoptive cellular therapy, in particular for cancer immunotherapy.

### BACKGROUND OF THE INVENTION

Adoptive Cellular Therapy (ACT, also referred as “Adoptive cell transfer”, “Adoptive cellular transfer”, or “Adoptive cell therapy”) is a promising anti-tumor immunotherapy for solid and hematologic malignancies. ACT employs the transfer of immunostimulatory cells that are recombinantly engineered using various approaches to re-direct a patient's immune response towards cancer cells, in particular by using genetically engineered T cells that express chimeric antigen receptor (CAR), now in late-phase clinical testing or approved (Cook K et al., 2018; Elahi R et al., 2018; Galluzzi L et al., 2018).

Recent literature provides different examples on how ACT can provide effective responses in metastatic cancers in comparison or in combination with other anti-cancer therapies, also to overcome the delayed relapses that are observed after an initial tumor regression while on continuous therapy. Immune escape in this settings appears associated to immunosuppressive mechanisms that protect the cells from immune recognition and elimination (Sharma P, et al., 2017). This problem poses a challenge to methods of treatment involving stimulation of an immune response, including ACT, and that may involve defective or down-regulated signaling pathways, such as those related to type I and/or II interferon receptor and JAK signaling, and directly involved in cancer biology (Bousoik E and Montazeri Aliabadi H, 2018).

Among other findings in this field, US20180051347 discloses that rarely occurring genetic mutations in the interferon receptor signaling pathway can result in lack of PD-L1 upregulation upon interferon exposure and result in innate resistance to PD-1 blockade immunotherapy. For those subjects who are consequently unlikely to respond to immunotherapies, including anti-PD-1 therapy, this finding enables the selection of a more appropriate, alternative treatment strategy (such as ACT). This method involves detecting a loss of function mutation or disruption in an interferon signaling pathway or a loss of function mutation or disruption in an MHC class I antigen presentation pathway, such as mutations that determine inactivation, deletion, or disruption of genes such Janus kinase 1 (JAK1), Janus kinase 2 (JAK2), or beta-2 microglobulin (B2M).

Intact tumor cell interferon (IFN) signaling was first identified as a critical piece of immune surveillance over two decades ago. More recently, experience with immune checkpoint blockade has validated the importance of tumor cell-intrinsic IFN signaling to anti-tumor immune responses in patients. Tumors from patients most likely to respond to immune checkpoint blockade are enriched for IFN gene signatures and genetic disruption of tumor IFN signaling can result in primary or acquired resistance to immune checkpoint blockade.

In primary resistance to immune checkpoint blockade, tumor cell defects in IFN signaling disrupt adaptive expression of PD-L1 and negate the effects of targeting the PD-1/PD-L1 axis. In acquired resistance, defects in IFN signaling render tumor cells insensitive to the positive effects of IFNs on antigen presentation and chemoattractant expression and the negative effects of IFNs on cell proliferation. However, whether intact tumor IFN signaling regulates the direct cytotoxic capacity of a tumor-specific T cell is less clear. Upon engaging their target through recognition of the MHC-antigen complex, tumor-specific T cells release granzyme and perforin which induce apoptosis of the target cell. The role of tumor-intrinsic interferon signaling in this context is particularly relevant for adoptive cell therapy approaches using tumor-specific T cells (e.g., TCR- or CAR-engineered T cell therapy).

Tumor intrinsic interferon signaling is central to the anti-tumor efficacy of T cells in the context of immune checkpoint blockade. However, also the response to ACT is not fully effective and is not yet known what biomarkers or drug combination can further improve or predict treatment outcome. Thus, there exists a need for methods and compounds that not only overcome microenvironments associated with malignant cells inhibiting effective immunotherapies, but also further improve clinical outcomes for those patients who not fully or only temporarily benefit from ACT.

### SUMMARY OF THE INVENTION

The present disclosure relates to the novel finding that nanoplexed formulations of a molecule acting as an agonist of Toll-Like receptor 3 (TLR3) and/or of any other cytoplastic double stranded RNA (dsRNA) sensors, such as RIG-I or MDA5, allow improving the response to ACT by overcoming type I and/or II interferon (collectively Interferon or IFN) signaling defects. These properties, in particular when the formulation comprises polyinosine:polycytidylic acid (also known as polyinosinic:polycytidylic acid, polyriboinosinic:polyribocytidylic acid, poly(I:C), poly(IC), pIC, or poly I:C) as TLR3, RIG-I PKR, and/or MDA5 agonist is administered intratumorally were not previously defined and exploited to improve therapeutic response to ACT and, in general, with respect to interferon signaling that affect the efficacy of agents that are used in cancer therapy. Accordingly, aspects of the disclosure relate to a method of treating a subject having cancer, comprising administering an Adoptive Cell Therapy in combination with a nanoplexed formulation of a TLR3, MDA5, and/or RIG-I agonist. The Adoptive Cell Therapy may comprise the administration of tumor-infiltrating lymphocytes, in vitro and/or ex vivo modified or sensitized immune cells, chimeric antigen receptor (CAR) cell therapy, and engineered T cell receptor (TCR) cell therapy

The current disclosure relates also to the use of nanoplexed formulations of a molecule acting as an agonist of Toll-Like receptor 3 (TLR3) and/or of any other cytoplastic double stranded RNA (dsRNA) sensors, such as Rig-I or MDA5, for treating any cancer that requires overcoming type I and/or II interferon signaling defects with or without the administration of a further immunotherapy. This use of nanoplexed formulations can be pursued after or, may be associated to, the evaluation of the cancer in the subject with respect to the presence or not of a loss of function mutation or disruption in an interferon signaling pathway or a loss of function mutation or disruption in an MHC class I antigen presentation pathway.

In some aspects, disclosed herein are methods of treating a subject having cancer, comprising: administering to the subject an Adoptive Cell Therapy in combination with a nanoplexed formulation of an agonist of a cytoplastic double stranded RNA (dsRNA) sensors, such as a TLR3, Rig-I, PKR, and/or MDA5 agonist. This combined administration can be performed simultaneously or sequentially, in particular by administering to the subject a nanoplexed formulation of a TLR3, Rig-I, PKR, and/or MDA5 agonist agonist at the time of or after having administered an Adoptive Cell Therapy. Moreover, this combined administration can be performed after or before having administered to the subject a further immunotherapy, in particular before or after having administered an anti-PD-1 therapy, an anti-PD-L1 therapy, or an anti-CTLA-4 therapy

In some embodiments, the agonist comprises poly(I:C). In a further embodiment, the agonist comprises RGC100. Other examples of TLR3 agonists include: double-stranded RNA, polyadenylic-polyuridylic acid (Poly(A:U)); polyinosine-polycytidylic acid high molecular weight (Poly(I:C) HMW); and polyinosine-polycytidylic acid low molecular weight (Poly(I:C) LMW). In some embodiments, said nanoplexed formulation is administered to the subject after having administered an adoptive cell therapy. This combined administration can be performed simultaneously or sequentially. In some embodiments, the nanoplexed formulation is administered to the subject prior to the adoptive cell therapy.

In some embodiments, the nanoplexed formulation of the TLR3, Rig-I, PKR, and/or MDA5 agonist and the Adoptive Cell Therapy are administered within 1 day of each other. In some embodiments, the nanoplexed formulation of the TLR3, Rig-I, PKR, and/or MDA5 agonist and the Adoptive Cell Therapy are administered within 1, 6, or 12 hours or within 1, 2, 3, 4, 5, 6, or 7 days or within 2, 3, 4, 5, 6, 7, 8, 9, 10, 11, or 12 weeks of each other (or any derivable range therein).

In some embodiments, the method further comprises administration of an additional therapy. In some embodiments, the subject has previously received an additional therapy or will receive an additional therapy. In some embodiments, the subject has been determined to be a non-responder to the additional therapy. In some embodiments, the subject has been determined to have a toxic response to the additional therapy. In some embodiments, the subject has not been administered a prior additional therapy. In some embodiments, methods of the disclosure exclude administration (or modify regimen and/or dosage) of an additional therapy.

In some embodiments, the additional therapy comprises a cytokine therapy, such as a therapy comprising the administration of an Interferon (such as Interferon beta) or an Interleukin (such as Interleukin-2). In some embodiments, the additional therapy comprises an immunotherapy, and in particular immune checkpoint blockade (ICB) therapy. In some embodiments, the ICB therapy comprises one or more of anti-PD-1 therapy, an anti-PD-L1 therapy, or an anti-CTLA-4 therapy. In some embodiments, the ICB therapy comprises ICB monotherapy. In some embodiments, ICB therapy comprises ICB combination therapy. In some embodiments, the ICB combination therapy comprises: (i) a PD-1, PDL1, or PDL2 inhibitor and (ii) a CTLA-4, B7-1, or B7-2 inhibitor. In some embodiments, the additional therapy comprises an additional therapy described herein.

It is specifically contemplated that any of the additional therapies may be excluded from embodiments of the disclosure. In specific embodiments, the subject has not been administered and is not prescribed an ICB therapy.

In some embodiments, ACT comprises the administration of one or more of tumor-infiltrating lymphocytes, in vitro and/or ex vivo modified or sensitized immune cells, chimeric antigen receptor (CAR) cell therapy, and engineered T cell receptor (TCR) cell therapy. The immune cells may be T cells or dendritic cells. In some embodiments, ACT involves the administration of cells that are genetically engineered with chimeric antigen receptor (CAR) or T Cell Receptor (TCR). Otherwise, ACT may be any other cell-based therapy directed to the treatment of cancer, using any type of primary cells or genetically modified cells (in particular immune cells).

In some aspects, the nanoplexed formulation of an agonist of a cytoplastic double stranded RNA (dsRNA) sensors, such as a TLR3, Rig-I, PKR, and/or MDA5 agonist is a complex formed by poly(I:C) molecules and an oppositely charged polyelectrolyte, in particular cationic polymers (or polycationic carries) including synthetic amino acid polymers (such as poly-L-lysine), lipofectamine, polyethyleneimine (PEI), natural DNA-binding proteins (such as histones), carbohydrate-based polymers such as chitosan, chemical variants or combinations known in the literature, and exemplified by formulation such as BO-112 and Poly-ICLC. In some embodiments, the nanoplexed formulation of a TLR3, Rig-I, PKR, and/or MDA5 agonist comprises a complex formed by poly(I:C) molecules and linear polyethyleneimine.

The administration of Adoptive Cell Therapy in combination with a nanoplexed formulation of a TLR3 agonist, with or without the administration of a further immunotherapy, is pursued after or, may be associated to, the evaluation of the cancer in the subject with respect to the presence or not of a loss of function mutation or disruption in an interferon signaling pathway or a loss of function mutation or disruption in an MHC class I antigen presentation pathway. In some embodiments, a biological sample from the subject has been evaluated for the presence or absence of a loss of function mutation or disruption in an interferon signaling pathway or a loss of function mutation or disruption in an MHC class I antigen presentation pathway. In some embodiments, a biological sample from the subject has been determined to have reduced MHC class I expression.

In some aspects, the loss of function mutation or disruption in an interferon signaling pathway is a mutation or disruption that truncates a Janus kinase 1 (JAK1) or a Janus kinase 2 (JAK2) protein, inactivates a JAK1 or a JAK2 protein, deletes a JAK1 or a JAK2 gene, or alters normal mRNA processing of a JAK1 or a JAK2 gene. In some aspects, the loss of function mutation or disruption in the interferon signaling pathway is a mutation or disruption that truncates a protein, inactivates a protein, or alters normal mRNA processing of a gene of at least one of: Interferon alpha/beta receptor 1 (IFNAR1), interferon gamma receptor 1 (IFNGR1), interferon gamma receptor 2 (IFNGR2), signal transducer and activator of transcription 1 (STAT1), signal transducer and activator of transcription 3 (STAT3), signal transducer and activator of transcription 5 (STATS), tyrosine kinase 2 (TYK2), interferon induced proteins with tetratricopeptide repeats (IFIT) genes, or interferon regulatory factor (IRF) genes. In particular, examples and lists of JAK1 and JAK2 mutations are summarized in US20180051347 and are regularly reported in the literature.

In some embodiments, the biological sample comprises a cancerous sample. In some embodiments, the biological sample comprises a sample obtained through biopsy of a suspected cancerous tissue. In some embodiments, the biological sample is one described herein.

In some embodiments, the nanoplexed formulation of a TLR3 agonist is administered by intratumoral injection. In some embodiments, the additional therapy comprises one or more of a chemotherapeutic agent, radiotherapy, an inhibitor of kinases, a cancer antigen vaccine, a MAPK targeted therapy, a mutant BRAF inhibitor, a MEK inhibitor, an ERK inhibitor, a Pan RAF inhibitor, an inhibitor of a metabolic enzyme, an oncolytic viral therapy, an anti-angiogenic therapy, a cGAS/STING pathway agonist, a cytokine (such an Interferon of an Interleukin), and an antibody against a cancer antigen.

In some embodiments, the cancer comprises recurrent cancer. In some embodiments, the cancer comprises stage I, II, III, or IV cancer. In some embodiments, the cancer comprises non-recurrent cancer.

In some aspects, present methods are applied or administered to subjects that have been diagnosed with cancer, in particular from a cancer that is PD-L1 positive (PD-L1⁺), at least prior to treatment with anti-PD-1 therapy, anti-PD-L1 therapy, or anti-CTLA-4 therapy. In some aspects, the cancer is PD-L1 negative (PD-L1⁻), with or without having been previously PD-L1⁺ cancer. In some aspects, the cancer is refractory to an antiPD-1 therapy, an anti-PD-L1 therapy, an anti-CTLA-4 therapy, or a combination thereof. In some aspects, the cancer is refractory to the anti-PD-1 therapy, the anti-PD-L1 therapy, the anti-CTLA-4 therapy, or the combination thereof. In some aspects, the anti-PD-1 therapy comprises an anti-PD-1 antibody, optionally wherein the antibody is nivolumab/BMS-936558/MDX-1106, pembrolizumab/MK-3475, pidilizumab/CT-011, or PDR001. In some aspects, the anti-PD-L1 therapy comprises an anti-PD-L1 antibody, optionally wherein the antibody is BMS-936559, MPDL3280A/atezolizumab, MSB00100718C/avelumab, or MEDI4736/durvalumab. In some aspects, the anti-CTLA-4 therapy comprises an anti-CTLA-4 antibody, optionally wherein the antibody is ipilimumab.

In some aspects, the methods for treating may involve further treating with an antibody against a cancer antigen, including CD19, CD20, CD22, CD25, CD38, CD52, CD137, CD138, CD254, CD261, CD262, CD309, CD319, CD326, VEGF, EGFR, LAG3, VISTA, and Her2/Her3.

In some aspects, the methods of the disclosure are used for treating a solid cancer or an hematologic malignances (e.g. acute lymphoblastic leukemia, acute myeloid leukemia, chronic lymphocytic leukemia, chronic myelogenous leukemia, hairy cell leukemia, multiple myeloma, AIDS-related lymphoma, (non-)Hodgkin lymphoma, and myeloproliferative neoplasms). In some embodiments, the cancer is solid cancer, such as an injectable or a cancer that that can be treated by intratumoral injection. Such cancer, in some embodiments, is selected from skin cancer (such as melanoma, skin cutaneous melanoma, dermatofibrosarcoma protuberans basal-cell skin cancer, squamous cell carcinoma, Merkel cell carcinoma, sebaceous carcinomas, keratoacanthoma, metastatic melanoma, or desmoplastic melanoma), endometrial cancer, kidney cancer, bladder cancer, breast cancer (such as breast carcinoma), prostate cancer (such as prostate adenocarcinoma), lung cancer (such as non-small cell lung cancer or lung adenocarcinoma), colon or colorectal cancer (such as colorectal adenocarcinoma), head and neck cancer, pancreatic cancer, genitourinary cancer, ovarian cancer, rectal cancer, gastric cancer, sarcoma, and esophageal cancer.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

Various implementations of the methods and compositions within the scope of the appended claims each have several aspects, no single one of which is solely responsible for the desirable attributes described herein.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-D: In vivo growth of tumors comprising different B16 murine melanoma cell lines (original or CRISPR-modified) tumors after adoptive cell transfer (ACT). Adoptive cell transfer (ACT) of gp100-specific pmel T cells was effective not only against wild type B16 tumors (B16 WT, gp100⁺; A) but also in B16 tumors lacking interferon signaling of either type I (B16 IFNAR1^{KO}; C) or type II (B16 JAK2^{KO}; B) compared to ACT with non-specific T cells (* means p<0.01, as calculated by repeated measures two-way ANOVA). This effect is not observed in B16 tumors lacking JAK1 (B16 JAK1^{KO}; D; NS, not significant).

FIG. 2A-B: Impact of BO-112 on in vivo growth of B16 WT tumors after pmel adoptive cell transfer (ACT). Intratumoral administration of BO-112 (versus vehicle, PBS) augmented the efficacy of adoptive cell transfer (ACT) of gp100-specific pmel T cells against B16 tumors (gp100⁺) in C57BL/6 mice (A; p<0.0022 and p<0.0001 at days 28 and 31 post-inoculation, respectively). This data are compared with two further control groups in which the administration of BL/6 T cells are used as control for ACT treatment (FIG. 2B).

FIG. 3A-C: Intratumoral (i.t.) nanoplexed poly(I:C) (BO-112) restores sensitivity of B16-JAK1(KO) tumors to pmel (gp100-specific) T cells in vivo. C57 BL/6 mice bearing subcutaneous B16 JAK1^{KO} tumors were treated 9 days post inoculation with ACT using pmel T cells or BL/6 T cells along with IL-2 (4×10⁶ T cells; IL-2 at 5×10⁴ IU/dose on days 9, 10, 11). Tumors were injected intratumorally with vehicle control (glucose 5%; B) or BO-112 (C; 50 μg/80 μL/dose on days 1 and 4 after ACT). In combination with BO-112, adoptive transfer of pmel T cells were effective against B16 JAK1^{KO} tumors compared to non-specific T cells in combination with BO-112 (mean difference 910.9 mm³, p<0.000001).

FIG. 4: BO-112 restores efficacy of ACT against B16-Jak1-KO tumors in an MHC-I dependent fashion. ACT using tumor-specific pmel T cells T cells along with IL-2 (4×10⁶ cells on day 9 after tumor implantation; IL-2 at 5×10⁴ IU/dose on days 9, 10, 11) is effective against B16-Jak1-KO tumors compared to ACT using non-specific BL/6 T cells when both groups are treated with intratumoral (i.t.) nanoplexed poly(I:C) (BO-112). However, despite intratumoral injection of BO-112, B16-B2m-KO tumors cells lacking a critical gene for MHC class I expression, do not respond to ACT using tumor-specific pmel (gp100-specific) T cells.

FIG. 5A-B: Antitumor activity of BO-112 in CRISPR/Cas9 B16 B2M^{KO} engineered melanoma cell lines (B16). C57 BL/6 mice bearing subcutaneous B16 B2M^{KO} tumors were treated with intratumoral injection of vehicle only (glucose 5%; A) or BO-112 (2.5 mg/kg; B) twice per week. Symbols and numbers identify individual mouse in each treatment group.

FIG. 6A-B: effect of intratumoral administration of BO-112 on the efficacy of dual immune checkpoint inhibitor blockade (anti-CTLA4 and anti-PD1) against wildtype B16 and B16-Jak1^{KO} tumors. In both models, BO-112 clearly increases the percentage of surviving animals, independently from dual immune checkpoint inhibitor blockade.

FIG. 7A-C: Cell surface expression of MHC-I and PD-L1 in wildtype B16 melanoma cells and B16 cells lacking different genes in presence of BO-112 or Type I/II interferons. Upon exposure vitro, BO-112 augments the expression of surface MHC-I and PD-L1 expression of the wildtype B16 tumor cell line, similar to the effects of type I and II IFNs (FIG. 7A, top panel). However, only BO-112, but not type I or type II IFNs, augments the expression of the B16-Jak1^{KO} cell line (FIG. 7A, bottom panel). The absence of Nlrc5 in two B16-Jak1-KO-Nlrc5-KO cell lines significantly abrogated IFNγ dependent MHC-I expression for wildtype B16 (without an effect on IFNγ dependent PD-L1 expression), but had not impact on the increased MHC-I expression in response to BO-112 (FIG. 7B). Thus, MHC-I induction by BO-112 occurs in both an IFN and Nlrc5 independent manner. The effect of BO-112 on surface MHC-I expression is also time and dose dependent; increases in MHC-I were observed at 24 hours, even after a three-hour pulse of 0.1 ug/mL of BO-112 (FIG. 7C).

FIG. 8A-B: BO-112 restores tumor-specific T cell recognition of B16-Jak1-KO tumors by inducing expression of MHC I genes. Tumor-specific IFNγ production by pmel T cells in wildtype B16 and B16-Jak1^{KO} tumor cells is measured after pre-treatment with either BO-112 or IFNγ (FIG. 8A). Expression of MHC I antigen processing machinery genes B2m and Tap1 in B16-Jak1^{KO} tumor cells is measured over 12 hours from exposure to BO-112 (FIG. 8B).

FIG. 9A-D: Effect of pattern recognition receptor (PRR) agonists on the cell surface expression of MHC-I in mouse and human cell lines. Unlike other PRR agonists (LPS, CpG, and a standard formulation of poly I:C), BO-112 augments MHC-I expression in the wildtype B16 mouse melanoma cells (A) and B16-Jak1-KO mouse melanoma cells (B). In a mouse macrophage cell line, BO-112 induces surface MHC-I expression to a greater extent than other PRR agonists (C). In the interferon-insensitive M202-JAK1^{KO} human melanoma cell line, both poly I:C and BO-112 (but not other PRR agonists) induce surface MHC-I expression (D).

FIG. 10: In vivo effect of BO-112 on tumors treated with ACT. RNA-sequencing analysis of B16-Jak1^{KO} tumors was performed five days after ACT with either control BL/6 T cells or tumor-specific pmel T cells, and with either intratumoral vehicle control or BO-112. We examined the 135 genes enriched in samples treated with BO-112 in combination with ACT using control BL/6 T cells and also ACT using pmel T cells. BO-112 induces an interferon-like signature highlighted by genes in the NF-kB pathway. A selection of 55 genes is categorized in three groups that are identified by a symbol (or their absence) and ranked by Log 2 fold change between vehicle control or BO-112 intratumoral injection.

FIG. 11: BO-112 induces MHC I expression in an interferon-independent, Nf-KB-dependent manner. B16-Jak1 KO cell line was treated with BO-112 in conjunction with a selective NF-kB inhibitor, BMS-345541. BMS-345541 abrogates the induction of MHC I by BO-112 in a dose-dependent manner (FIG. 11A). A transient knockdown of Rela via two different siRNAs achieved a similar effect, inhibiting the upregulation of surface MHC I by BO-112 in B16-Jak1^{KO} tumors (B), as well as in M202 JAK1-KO human melanoma cell line (C).

FIG. 12: NF-kB activation by BO-112 is dependent on PKR, an upstream dsRNA sensor. In both B16-WT and B16-Jak1-KO cell lines, BO-112 results in nuclear translocation of Nf-KB p65 subunit, according to Western Blot analysis (FIG. 12A; control vehicle is indicated as Gluc. 5%). This nuclear translocation of Nf-KB p65 in response to BO-112 is diminished in the context of siRNA targeting PKR and not by control siRNA (indicated as siNTC Likewise, BO-112 (indicated as BO) induces expression of key MHC I gene, TAP1, in B16-WT and B16-Jak1-KO cell lines, which is abrogated by siRNA against PKR (FIG. 12B; statistical significance is indicated by * and **).

### DETAILED DESCRIPTION OF THE INVENTION

The current disclosure provides methods to overcome some types of resistance to anticancer drugs and in particular interferon-mediated tumor resistance, as tested in a murine model of melanoma using a nanoplexed formulation of poly I:C (BO-112) that activates TLR3, MDA5, and/or RIG-I. This approach is exemplified in genetically modified B16-F10 melanoma cell lines that lacked genes necessary for type I and II interferon signaling, showing as well the involvement of protein kinase RNA-activated (PKR). Because interferon-signaling regulates antigen presentation in such tumor model, the ability the induce WIC Class I expression independent of interferon in presence (or not) of BO-112, was evaluated in cell lines either in vitro or after being and injected in vivo, in combination (or not) with model cells for Adoptive Cell Therapy (ACT; pmel T-cells). Using this assay to determine the efficacy of ACT in tumors lacking interferon signaling, combination of BO-112 and pmel ACT was identified a very promising regimen.

Briefly, and as described in more detail below, described herein are improved methods for treating a subject with cancer by ACT that, by administering a nanoplexed formulation of an agonist of a cytoplastic double stranded RNA (dsRNA) sensors, such as a TLR3 agonist, allow not only improving the therapeutic response to ACT but overcoming type I and/or II interferon signaling defects that affects ACT efficacy. These mutations can be identified by using known technologies such as sequencing assays such as Sanger sequencing or next generation sequencing. The sequencing assay may further comprises prior target amplification by PCR. In some aspects, NGS comprises whole-exome sequencing, whole-genome sequencing, de novo sequencing, phased sequencing, targeted amplicon sequencing, or shotgun sequencing. In some aspects, the determining step further comprises experimentally determining an RNA profile status of the mutation. In some aspects, the experimentally determining the RNA profile status comprises RNA-Seq or a qPCR assay prior target amplification by PCR.

Methods of treating a subject having cancer are described herein in more detail. Also described herein are methods of assessing a subject having cancer. In particular, present disclosure also provides methods of treating a subject having cancer that may be defined also with respect to methods of assessing different clinical criteria or parameters in a subject having cancer. In particular, the combined administration of ACT and nanoplex formulation of a TLR3, RIG-I, PKR, and/or MDA5 agonist (possibly through PKR signaling) can follow the analysis of criteria related to the response to cancer immunotherapy (such as anti-PD-1 therapy, anti-PD-L1, or anti-CTLA-4 therapy, alone or in any combination thereof) using clinical reports or biological samples from the patients. In addition to the identification of mutation in interferon signaling pathways, criteria such as qualitative and/or quantitative feature of immune cells (such as NK cells, B cells, CD4+/CD8+ T cells) where changes in specific cell markers and (sub-) populations can be measured from blood samples, and/or within the primary tumor cells from patients that are transferred and analyzed for growth kinetics or drug sensitivity in animal models. These evidences may be useful for the selection of patients that would particularly benefit from combined administration of ACT and the nanoplex formulation of a TLR3, RIG-I, PKR, and/or MDA5 agonist, in view of mutational burden or other molecular signatures of tumor, immune profile of the subject, and/or other appropriate clinical criteria. ACT and the nanoplex formulation of a TLR3, RIG-I, PKR, and/or MDA5 agonist can be also tested and in combination with standard-of-care, conventional treatments (such as radiotherapy, chemotherapy, inhibitors of cellular kinases, cytokines, etc.) or treatments involving novel mechanisms and/or novel candidate anti-cancer drugs that are compatible with such combined administration of ACT and the nanoplex formulation of a TLR3, RIG-I, and/or MDA5 agonist. The standard-of-care treatment can be administered before or after administered the methods of the disclosure.

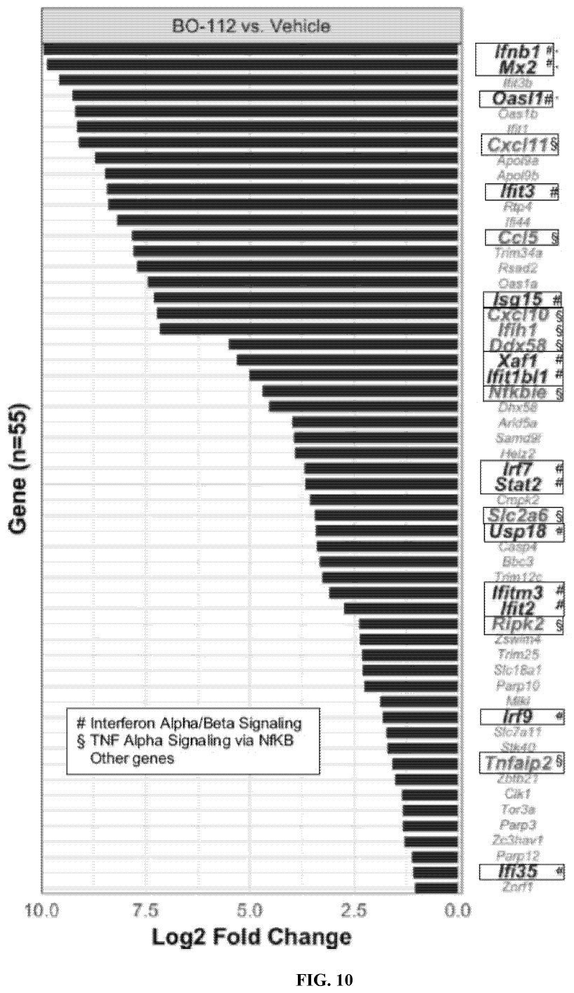

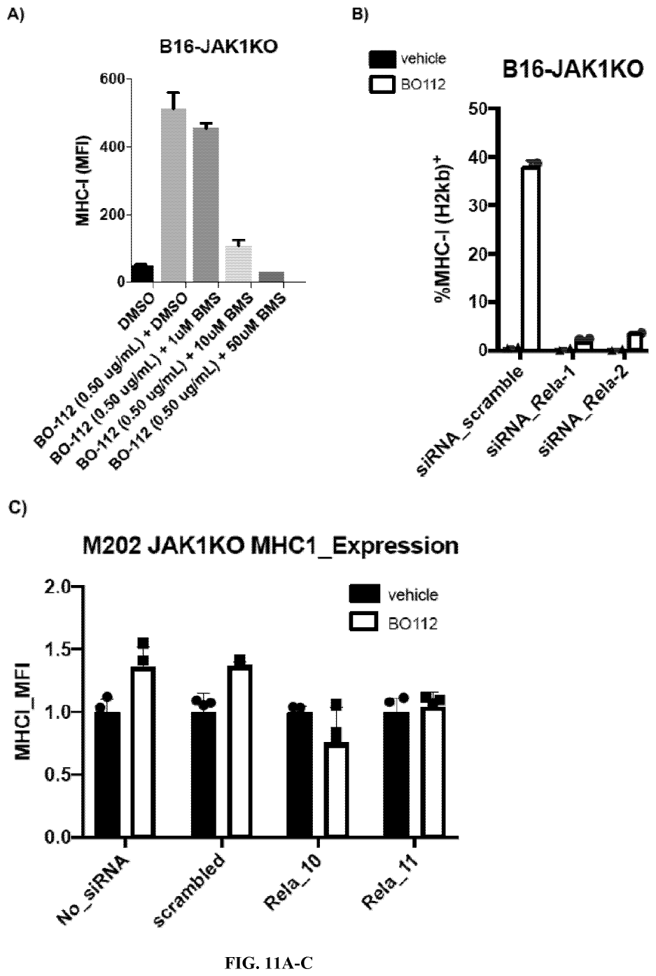

ACT and the nanoplex formulation of a TLR3, RIG-I, PKR, and/or MDA5 agonist may be also administered for treating cancer types which have not previously demonstrated any sensitivity to immunotherapy, such as those have a high mutational burden (or microsatellite instability), as detected by gene sequence and/or expression profiling that affect interferon-dependent responses and/or immune cells-mediated response of therapeutic relevance within cancer or in tumor microenvironment, as also shown in FIG. 10. For example, the combined administration of ACT and nanoplex formulation of a TLR3, RIG-I, PKR, and/or MDA5 agonist may be exploited for promoting therapeutically relevant events such as tumor cell death, enhanced local and/or systemic T cell immune response either directly (within injected tumors) or in distant tumors, and other mechanisms that may be useful for treating cancers that are recurrent, unresponsive or refractory to other therapies, and that may be resensistize the tumor to cancer immunotherapies and/or ACT.

### I. Definitions

Terms used in the claims and specification are defined as set forth below unless otherwise specified.

The term “interferon signaling pathway” refers to any part of either the type-I interferon (interferon α or β) or the type-II interferon (interferon γ) signaling networks, including, but not limited to, receptors, kinases, transcription factors, genes regulated by interferon signaling, positive or negative regulators of interferon signaling.

The term “MHC class I antigen presentation pathway” refers to any gene involved in the processing or presenting of antigenic peptides on MHC class I molecules. Genes involved in the pathway include, but are not limited to, components of MHC class I molecules, components of the peptide-loading complex, and components of the immuno-proteosome.

The term “immune checkpoint therapy” or “immunotherapy” refers to therapies that stimulate a subject's immune own system to target disease, including cancer. Many immunotherapies work through inhibiting various immune checkpoints that limit activation of the immune system, thus in turn allows activation of the immune system.

The term “PD-L1+” refers to a sample, including a cancer tissue sample or biopsy, that is positive for the marker PD-L1. The sample can be determined to be positive for the marker PD-L1 by immunohistochemistry, immunostaining, RT-qPCR, RNA-Seq, or any other method known to those skilled in the art.

The term “refractory” (and variations thereof such as “not responding”, “unresponsive” or “resistant”) refers to a state of a disease, such as cancer, where the disease is no longer responsive to a given treatment. In some instances, the disease may have previously been responsive to the given treatment but is no longer responsive. In some instances, the disease may be refractory to a given treatment due to mutations.

The term “nanoplexes” or “nanoplex formulation” (also named “polyplex nanoparticles”) means drug nanoparticle with an oppositely charged polyelectrolyte (Kadam, R N et al., 2015). Nanoplex formulation is characterized through the production yield, complexation efficiency, drug loading, particle size and zeta potential using scanning electron microscopy, differential scanning calorimetry, Dynamic Light Scattering (DLS), or X-ray diffraction. Additional features are defined with respect to the component of the particles comprised in the nanoplex formulation of TLR3 agonist, or the composition itself (preferably an aqueous composition or other injectable composition) such as the size and/or and concentration of poly(I:C) molecules in the composition, the type of polymer carrier, the average size, median diameter, the polydispersity index and/or mono-modal distribution of particles, or the pH and osmolarity of the composition. Moreover, this composition may further comprise at least one pharmaceutically acceptable carrier, organic solvent, excipient and/or adjuvant that is appropriate for the methods of the disclosure.

In particular, the particles in the nanoplex formulation comprises a complex of polyinosinic-polycytidylic acid [poly(I:C)], or a salt or solvate thereof, wherein at least 40% of poly(I:C) molecules comprised in said particles have at least 850 base pairs, and at least 50% of poly(I:C) molecules comprised in said particles have between 400 and 5000 base pairs, with a water-soluble, linear homo-polyalkyleneimine or hetero-polyalkyleneimine (preferably, linear polyethyleneimine, or LPEI), or a salt and/or solvate thereof, wherein the average molecular weight of said linear polyalkyleneimine is between 17 and 23 kDa; at least 90% of said particles have a mono-modal diameter distribution below 300 nm; have a z-average diameter of 80+/−20 nm, as measured according to ISO 22412 (2017 version, or as later amended), with polydispersity index of said particle diameter which is inferior to 1.5; have a median diameter (D50%) of 85+/−20 nm; and are comprised in a composition contains polyinosinic-polycytidylic acid [poly(I:C)] at a concentration of at least 0.5 mg/mL, with a pH of between 2 and 4 and osmolality of between 200 and 600 mOsm/kg and a zeta potential between 35 mV and 50 mV, according to ISO 13099-2 (2012 version, or as later amended).

In particular, exemplary nanoplexes comprising poly(I:C) molecules as TLR3, RIG-I, PKR, and/or MDA5 agonist are disclosed under the names of BO-110 (Tormo D et al., 2009), BO-112 (WO2017085228; PCT/EP2017/079688, published as WO2018210439), Poly-ICLC and similar products based on polyriboinosinic:polyribocytidylic acid (Patel M C et al., 2014). In particular, such formulation, when prepared as described in WO2017085228 or PCT/EP2017/079688, may provide additional properties of therapeutic interest, either with respect to the activation of other proteins known as dsRNA sensors (such as MDA-5 and/or RIG-I) or to the increased secretion of chemokines, cytokines, or other secreted proteins. These properties may be identified at the level of either tumor cells or immune cells (such as T cells, dendritic cells, macrophages, and the like) that are exposed to the nanoplexes comprising poly(I:C) molecules that are agonists of dsRNA sensors, such as a TLR3, RIG-I, PKR, and/or MDA5 agonist following the nanoplexes administration, e.g. by injection. Such nanoplexes are preferably administered by intratumoral injection in order to provide a specific effect upon interferon receptor signaling in either cancer cells or tumor-infiltrating cells within tumor and amplify therapeutic effects of ACT, before or after determining the presence of mutations and/or administration of anti-PD-1 therapy, anti-PD-L1 therapy, or anti-CTLA-4 therapy. WO2017085228 describes additional embodiments that may be used in the methods and formulations of the disclosure and is specifically incorporated by reference for all purposes.

The term “cancer” refers to all neoplastic cell growth and proliferation, whether malignant or benign, and all pre-cancerous and cancerous cells and tissues. The terms “cancer,” “cancerous,” “cell proliferative disorder,” “proliferative disorder”, “tumor”, and “carcinoma”, are not mutually exclusive as referred to herein. In particular, the term “solid tumor” refers to an abnormal mass of tissue that usually does not contain cysts or liquid areas. Solid tumors may be benign or malignant. Different types of solid tumors are named for the type of cells that form them. Examples of solid tumors are sarcomas (including cancers arising from transformed cells of mesenchymal origin in tissues such as cancellous bone, cartilage, fat, muscle, vascular, hematopoietic, or fibrous connective tissues, carcinomas (including tumors arising from epithelial cells), carcinomas of solid tissue, squamous cell carcinomas of the mouth, throat, larynx, cancer of the endocrine system, cancer of the thyroid gland, adenomas, melanomas, lymphomas, mesothelioma, neuroblastoma, retinoblastoma, and nervous system cancers, and benign lesions such as papillomas and the like.

As used herein, the term “combination therapy” refers to those situations in which a subject is simultaneously exposed to two or more therapeutic regimens, such as exposure to two or more therapeutic agents. In some embodiments, two or more agents may be administered simultaneously. Alternatively, such agents may be administered sequentially; otherwise, such agents are administered in overlapping dosing regimens. In particular, ACT may be administered before treatment with the treatment with the nanoplex formulation of a TLR3 agonist (and before any other cancer therapy). Alternatively, ACT may be administered after treatment with the treatment with the nanoplex formulation of a TLR3, RIG-I, PKR, and/or MDA5 agonist (and before any other cancer therapy).

The term “sequentially” as used herein means that ACT and the nanoplex formulation of a TLR3, RIG-I, PKR, and/or MDA5 agonist are administered with a time separation of more than about 10 minutes, 20, minutes, 30 minutes, or 60 minutes. For example, the time between the sequential administration of ACT and the nanoplex formulation of a TLR3, RIG-I, PKR, and/or MDA5 agonist can be more than about 60 minutes, more than about 2 hours, more than about 5 hours, more than about 10 hours, more than about 1 day, more than about 2 days, more than about 3 days, or more than about 1 week apart. The optimal administration times may depend on the rates of metabolism, excretion, and/or the pharmacodynamic activity of ACT and the nanoplex formulation of a TLR3, RIG-I, PKR, and/or MDA5 agonist.

The term “simultaneously” as used herein, means that ACT and the nanoplex formulation of a TLR3, RIG-I, PKR, and/or MDA5 agonist are administered with a time separation of about 10 minutes or less, such as no more than 5 minutes, or no more than about 1 minute. Administration of the ACT and the nanoplex formulation of a TLR3, RIG-I, PKR, and/or MDA5 agonist can be by simultaneous administration of a single formulation (e.g. a formulation comprising ACT and the nanoplex formulation of a TLR3, RIG-I, PKR, and/or MDA5 agonist) or of separate formulations (e.g., a first formulation including ACT and a second formulation including the nanoplex formulation of a TLR3, RIG-I, PKR, and/or MDA5 agonist).

As used herein, the term “patient” or “subject” refers to any organism to which a provided composition is or may be administered, for example, for experimental, diagnostic, prophylactic, and/or therapeutic purposes. Typical patients include animals including but not limited to mammals such as mice, rats, rabbits, non-human primates, and/or humans. In some preferred embodiments, a patient is a human. In some embodiments, a patient is suffering from or susceptible to one or more disorders or conditions. A patient may display one or more symptoms of a disorder or condition, or may have been diagnosed with one or more disorders or conditions (such as cancer, or presence of one or more tumors). In some embodiments, the patient is receiving or has received certain therapy to diagnose and/or to treat such disease, disorder, or condition with an appropriate dosing regimen.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length, alternating the administration of two elements of the combination of the disclosure, if appropriate. Alternatively, a dosing regimen comprises a plurality of doses and at least two different time periods separating individual doses. In some embodiments, all doses within a dosing regimen are of the same unit dose amount. Alternatively, different doses within a dosing regimen are of different amounts. In some embodiments, a dosing regimen comprises a first dose in a first dose amount, followed by one or more additional doses in a second dose amount different from the first dose amount. A dosing regimen may comprise a first dose in a first dose amount, followed by one or more additional doses in a second dose amount same as the first dose amount and it is correlated with a desired or beneficial outcome when administered across a relevant population, namely a therapeutic dosing regimen. In some aspects, the dosing regimen may include also sub-therapeutic doses, for example, when the administered dose of either ACT and/or the nanoplex formulation of a TLR3, RIG-I, PKR, and/or MDA5 agonist is lower than what it would be in a monotherapy of each component of the combination.

The term “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

The term “therapeutically effective amount” is an amount that is effective to ameliorate a symptom of a disease. A therapeutically effective amount can be a “prophylactically effective amount” as prophylaxis can be considered therapy.

The term “sub-therapeutic dose or amount” means that a dose or amount of a pharmacologically active substance (i.e. ACT or the nanoplex formulation of a TLR3, RIG-I, PKR, and/or MDA5 agonist) is below the dose or amount of that substance that is administered, as the sole substance, to achieve a therapeutic effect. The sub-therapeutic dose of such a substance may vary depending upon the subject and disease condition being treated, the weight and age of the subject, the severity of the disease condition, the manner of administration and the like, which can readily be determined by one of ordinary skill in the art. In one embodiment, the sub-therapeutic dose or amount of the substance is less than 90% of the approved full dose, such as that provided in the U.S. Food & Drug Administration-approved label information for such substance. In other embodiments, the sub-therapeutic dose or amount of the agent is less than 70%, 50%, 30%, or even 10% of the approved full dose, such as from 10% to 90%, 30% to 70%, 50% to 90%, or another range within the values provided herein.

The term “administered” or administration” refers to the administration of a composition to a subject, in particular of a therapeutically effective amount of a pharmaceutical composition. Administration to an animal subject, such as a human, can be accomplished via a plurality of routes. Conventional and pharmaceutically acceptable routes of administration include, but are not limited to, intratumoral, bronchial (including by bronchial instillation), buccal, enteral, intra-arterial, intranodal, intradermal, intragastric, intramedullary, intramuscular, intranasal, intraperitoneal, intrathecal, intravenous, intraventricular, within a specific organ (for example, intrahepatic), mucosal, nasal, oral, rectal, subcutaneous, sublingual, topical, tracheal (including by intratracheal instillation), transdermal, vaginal, or vitreal. Administration may also involve intermittent dosing. Alternatively, administration may be by continuous dosing (e.g., perfusion) for at least a predetermined period of time. As is known in the art, antibody therapy is commonly administered parenterally, e.g. by intravenous, subcutaneous, or intratumoral injection, for instance, particularly when high doses within a tumor are desired). Routes of administration can be combined, if desired, or adjusted depending upon the disease. Route of administration primarily will depend on the nature of cancer being treated and the type of response that is desired.

The term “treating” (and variations thereof such as “treat” or “treatment”) refers to clinical intervention in an attempt to alter the natural course of a disease or condition in a subject in need thereof. Treatment can be performed both for prophylaxis and during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

The term “mutation” refers to an alteration in the nucleotide sequence of a subject's genome. Mutations may affect the coding region of a gene and include, but are not limited to, a missense mutation causing a substitution from one amino acid to another, a nonsense mutation causing a substitution from an amino acid to a stop codon, or a frameshift mutation causing a change in the frame of the protein translated. A mutation may result in the truncation of a protein, wherein the full-length protein is not expressed. A mutation may result in the inactivation of a protein, wherein the protein can no longer perform the full activity of the wild-type protein. A mutation may be in a non-coding region of a gene and include, but are not limited to, mutations in promoter elements, 5′ untranslated regions (5′-UTR), 3′ untranslated regions (3′-UTR), and introns. A mutation may result in an alteration of the normal RNA processing, such as improper RNA splicing, nonsense mediated decay, non-stop decay, or no-go decay. A mutation may alter the RNA expression level of a gene. A mutation may be a point mutation, wherein there is a single nucleotide difference. A mutation may be an insertion, deletion, or alteration of more than one nucleotide. The term “loss of function mutation” refers to a mutation that results in a gene product no longer being able to perform its normal function or its normal level of activity, in whole or in part. Loss of function mutations are also referred to as inactivating mutations and typically result in the gene product having less or no function, i.e., being partially or wholly inactivated.

The term “loss of function disruption” refers to an alteration that results in a gene product no longer being able to perform its normal function or its normal level of activity, in whole or in part. Loss of function disruptions include epigenetic silencing. Epigenetic silencing refers to non-mutational gene inactivation that can be propagated from precursor cells to clones of daughter cells. The addition of methyl groups to cytosine residues in CpG dinucleotides in DNA is an exemplary biochemical modification that meets this requirement.

The term “ameliorating” (and variations thereof such as “ameliorate”, “amelioration”) refers to any therapeutically beneficial result in the treatment of a disease state, e.g., a cancerous disease state, including prophylaxis, lessening in the severity or progression, remission, or cure, in general or with respect to either a prior treatment (including ACT and/or immunotherapies, such as anti-PD-1 therapy) to which a subject was partially or fully resistant or not responding.

As used herein, the terms “biological sample” or “sample” typically refers to a sample obtained or derived from a biological source of interest, for instance, a tissue or organism or cell culture. One source of interest can be an animal or a human organism. The biological sample may comprise one or more biological tissues or fluids, but preferably it is blood or plasma.

### II. Nanoplexed Formulations

The current disclosure relates to specific uses and methods of using nanoplexed formulations, such as a composition as described in WO2017085228 and PCT/EP2017/079688, whose disclosure is summarized in this section, where the nanoplexed formulations are collectively identified as BO-11X and exemplified by the BO-112 formulation. In some embodiments, the nanoplexed formulation is a composition comprising particles wherein (i) each of said particles comprises a complex of at least one double-stranded polyribonucleotide, or a salt or solvate thereof, and at least one polyalkyleneimine, or a salt and/or solvate thereof; (ii) at least 95%, or at least 90%, of said particles has a diameter of less than or equal to 600 nm, preferably, less than or equal to 300 nm (for example, between 140 and 250 nm); and (iii) said particles have a z-average diameter of less than or equal to 200 nm, preferably less than or equal to 150 nm, in particular, as measured according to ISO 22412.

In a preferred embodiment, the nanoplexed formulation is an aqueous composition comprising particles wherein (i) each of said particles comprises a complex of at least one double-stranded polyribonucleotide, or a salt or solvate thereof, and at least one linear polyalkyleneimine, or a salt and/or solvate thereof, wherein said double-stranded polyribonucleotide is polyinosinic-polycytidylic acid [poly(l:C)] and the average molecular weight of said linear polyalkyleneimine is between 17 and 23 kDa; (ii) at least 90% of said particles has a mono-modal diameter distribution below 300 nm; (iii) said particles have a z-average diameter of less than or equal to 150 nm, as measured according to ISO 22412; and (iv) said composition has a zeta potential equal or superior to 30 mV, according to ISO 13099.

The nanoplexed formulation is preferably in the form of an aqueous composition comprising particles as disclosed herein wherein: (i) each of said particles is formed by making a complex of at least one double-stranded polyribonucleotide, or a salt or solvate thereof, and at least one linear polyalkyleneimine, or a salt and/or solvate thereof, wherein said double-stranded polyribonucleotide is polyinosinic-polycytidylic acid [poly(l:C)] and the average molecular weight of said linear polyalkyleneimine is between 17 and 23 kDa; (ii) at least 90% of said particles has a mono-modal diameter below 300 nm; (iii) said particles have a z-average diameter of less than or equal to 150 nm, as measured according to ISO 22412; and (iv) said composition has a zeta potential equal or superior to 30 mV, according to ISO 13099; wherein said particles are formed at the ratio of the number of moles of nitrogen of said polyalkyleneimine to the number of moles of phosphorus of said double-stranded polyribonucleotide in said composition being equal to or greater than 2.5.

The nanoplexed formulation can be a composition obtainable by lyophilisation of the aqueous composition as disclosed herein.

In some embodiments, the nanoplexed formulation comprises particles wherein: (i) each of said particles comprises a complex of at least one double-stranded polyribonucleotide, or a salt or solvate thereof, and at least one polyalkyleneimine, or a salt and/or solvate thereof, wherein (a) said double-stranded polyribonucleotide is polyinosinic-polycytidylic acid [poly(l:C)], wherein at least 60% of said double-stranded polyribonucleotides have at least 850 base pairs, at least 70% of said double-stranded polyribonucleotides have between 400 and 5000 base pairs, and between 20% and 45% of said double-stranded polyribonucleotides have between 400 and 850 base pairs; and (b) said polyalkyleneimine comprises at least 95% polyethyleneimines, wherein the weight average molecular weight of said polyalkyleneimine is between 17 and 23 kDa and the polydispersity index is <1.5, and wherein the ratio of the number of moles of nitrogen of said polyalkyleneimine to the number of moles of phosphorus of said double-stranded polyribonucleotide in said composition is between 2.5 and 5.5; (ii) at least 99% of said particles has a diameter of less than or equal to 600 nm; and (iii) said particles have a z-average diameter of between 30 nm and 150 nm.

In some embodiments, (i) each of said particles comprises a complex of at least one double-stranded polyribonucleotide, or a salt or solvate thereof, and at least one polyalkyleneimine, or a salt and/or solvate thereof, wherein (a) said double-stranded polyribonucleotide is polyinosinic-polycytidylic acid [poly(l:C)], wherein at least 60% of said poly(l:C) has at least 850 base pairs, at least 70% of said poly(l:C) has between 400 and 5000 base pairs, and between 20% and 45% of said poly(l:C) has between 400 and 850 base pairs; and (b) said polyalkyleneimine is polyethyleneimine (PEI), wherein the weight average molecular weight of said PEI is between 17.5 and 22.6 kDa and the polydispersity index is <1.5, and wherein the ratio of the number of moles of nitrogen of said polyalkyleneimine to the number of moles of phosphorus of said double-stranded polyribonucleotide in said composition is between 2.5 and 4.5; (ii) at least 99% of said particles has a diameter of less than or equal to 500 nm; (iii) said particles have a z-average diameter of between 60 nm and 130 nm; and (iv) said particles have a median diameter (D50%) of between 75 nm and 150 nm.

The particles that are made of and formed by said complexes may present additional features, as per the disclosure below, such that in further embodiments said particles may comprise further components such as excipients like mannitol or glucose, or the absence of further elements, such as cancer-targeting functionality or other moieties and linkers. Additional features can be defined in further preferred embodiments when the particles are provided and analysed within the compositions [i.e. within the liquid (aqueous) or lyophilised formulations], such as when defined as having a mono-modal size distribution within specific ranges, for example, between 30 nm and 150 nm, or when the composition is characterised by the absence of single-stranded polyribonucleotide molecules (as established by a low or absent hyperchromic effect). Other features as defined in accordance to internationally established standards that are required for regulatory approval and/or Good Manufacturing Processes are disclosed in WO2017085228 and PCT/EP2017/079688.

In one embodiment, the nanoplexed formulation comprises polyinosinic-polycytidylic acid [poly(l:C)] molecules. Said double-stranded polyribonucleotide molecules comprise strands of, for example, poly(l) that pair with poly(C), thus forming double-stranded polyribonucleotides, wherein each strand may comprise up to 5% of ribonucleotides different from the majority of ribonucleotides in said strand and/or comprise up to 5% mismatched base pairs, more preferably up to 1% of ribonucleotides different from the majority of ribonucleotides in said strand, and/or comprise up to 1% mismatched base pairs. Depending on the selected polyribonucleotide and/or the process for generating said complexes, a fraction of the polyribonucleotides comprised in the complex may also comprise single-stranded (i.e. non-paired) polyribonucleotides.

In some embodiments, the nanoplexed formulation comprises particles having a mono-modal diameter distribution, in particular within the sub-micrometer range indicated above. Indeed, in one aspect the aqueous composition of the present disclosure comprises particles wherein at least 90% of said particles has a mono-modal diameter distribution below 300 nm, wherein said particles have a z-average diameter of less than or equal to 150 nm, as measured according to ISO 22412. Particles (or their aggregates) having a size superior to such values (e.g. in the micrometer range, such as above 10μη) that may be still present (but, in any case below the limits indicated in European Pharmacopoeia) can be removed by filtration, at the end of manufacturing and/or just before administration (for example, through 0.8 micrometer filter). Thus, all or the large majority of particles comprised in this composition may present a mono-modal diameter distribution within the composition that, as shown in the Examples, is established during their preparation and can be maintained and adapted according to the desired use and/or storage.

In another preferred embodiment, at least 95% or 90% of particles in the nanoplexed formulation has a diameter of less than or equal to 600 nm (i.e. the maximum particle diameter below which 95% or 90% of sample intensity falls=D95% or D90%=600 nm), more preferably not exceeding the diameter of 500 nm, still more preferably not exceeding the diameter of 400 nm, and yet more preferably not exceeding the diameter of 300 nm. Within such limits, Even more preferably, at least 99% of said particles has a diameter of less than or equal to 600 nm, yet more preferably at least 99% of said particles has a diameter of less than or equal to 500 nm, much more preferably at least 99% of said particles has a diameter of less than or equal to 400 nm and yet more preferably not exceeding the diameter of 300 nm. On the other hand, in a preferred embodiment, said particles have a median diameter (D50%) between 75 and 150 nm, more preferably between 80 and 130 nm, and a D90% of between 140 and 250 nm, more preferably between 170 and 240 nm.

In another preferred embodiment of the nanoplexed formulation, the particles in the nanoplexed formulation have a z-average diameter below 150 nm, and more preferably in ranges comprised between 30 nm and 150 nm (such as furthermore preferably between 50 nm and 150 nm, between 75 nm and 150 nm, between 50 nm and 100 nm, between 100 nm and 150 nm, or between 60 nm and 130 nm). More preferably, said particles of the aqueous composition of the present disclosure have a mono-modal diameter distribution between 30 nm and 150 nm.

The nanoplexed formulations can be provided as compositions further comprising a pharmaceutically acceptable carrier, excipient, organic solvent, and/or adjuvant such as glycerol, ethanol, glucose or mannitol, preferably glucose or mannitol, more preferably in a concentration of between 1 and 10% (weight/volume)] [i.e. wherein said composition is formed by additionally adding glucose or mannitol in a concentration of between 1 and 10% (weight/total volume of said composition)] that is best adapted to the preferred final form (such as liquid or lyophilised), uses, shipment, storage, administration with other compounds, and/or further technical requirements. In a more preferred embodiment, said composition further comprises at least one compound selected from an organic compound, an inorganic compound, a nucleic acid, an aptamer, a peptide or a protein.

These compostions are particularly adapted for direct administration to the cancer cells, for example by means of intratumoral or peritumoral injection into skin or an internal organ or tissue comprising such tumors and cancer cells. In a preferred embodiment of the present disclosure, said medicament is injectable. In a more preferred embodiment of the present disclosure, said medicament is an injectable, aqueous composition, optionally comprising a pharmaceutically acceptable carrier, excipient and/or adjuvant. This injectable, aqueous composition can be provided as such or after diluting a concentrated preparation of poly(l:C) molecules (at a respective concentration of at least 0.5 mg of poly(l:C)/ml of the total volume of composition to be made, or more, as established when preparing the particles in terms of the respective weight of poly(l:C) molecules that are added to a given volume of solution) or a lyophilised composition in order to make up a total volume of the composition of the disclosure. This means that said composition is provided in the foregoing concentrations determined in terms of the weight of poly(l:C) or poly(A:U) employed in making the complex per volume of the total aqueous composition, but may be concentrated where appropriate, especially for long-term storage and/or intratumoral administration. In particular, the BO-11X formulation with double-stranded poly(l:C) molecules at such high concentrations (i.e. that made from particles comprising a complex formed by complexing at least 0.5 mg up to 0.7 mg, preferably 0.9 mg, more preferably 2.0 mg or more, of poly(l:C) with linear PEI per mL of the total aqueous composition) is most appropriate for administration and use as a medicament. The intratumoral or peritumoral injection of such a composition (depending also on the actual accessibility and/or size of the tumor mass as evaluated by the practitioner) in one or more small or restricted locations where tumors and cancer cells are present, may provide a stronger and/or more timely therapeutic effect.

In another preferred nanoplexed formulation the double-stranded polyribonucleotides are poly(I:C) molecules that are present in the BO-11X formulations result from the annealing of polyinosinic acid [poly(I)] molecules and polycytidylic acid [poly(C)] single-stranded molecules that have themselves specific ranges of percentages for sizes below 0.4 Kb, between 0.4 Kb and 0.85 Kb, between 0.85 Kb and 5.0 Kb, and above 5.0 Kb which also provides means for generating an aqueous solution of poly(I:C) molecules (already containing or not an excipient such as glucose or mannitol) and have appropriate features for being mixed with aqueous solution of a polyalkyleneimine (such as polyethyleneimine) for producing the BO-11X formulations. The poly(I:C)-containing formulation resulting from mixing these two aqueous solutions is then maintained as a batch preparation (preferably still as a aqueous solution or in a lyophilized form) or can be directly prepared in aliquots, each contained in a single-use vials, syringes, or other appropriate container for storage, single use of such aliquots, and/or lyophilisation. BO-11X formulations (in a liquid or lyophilized form) can be stored at room temperature or a temperature below 0° C. or below −20° C.

In preferred, alternative nanoplexed formulations, further compounds (such as one or more antibody, hormone, peptide, cytokine, excipient, carrier, inhibitor of an enzymatic activity, chemotherapeutic agent, antibiotic, stabilizing agent, labelling agent, organic solvent, preservatives, carriers, or other drug) can be either added in each of the two aqueous solutions (if not altering the correct formation of the particles or any other of the features listed above for BO-11X formulations) prior to their mixing or after that BO-11X formulation has been produced by mixing the two aqueous solutions (of double-stranded polyribonucleotide and polyalkyleneimine). Such additional components that are consequently administered at the same time with BO-11X components can provide a composition with improvements in the bioavailability, efficacy, pharmacokinetic/pharmacodynamic profiles, stability, metabolization, or other property of pharmaceutical interest that are not observed when each of initial BO-11X formulation or the additional component (another compound of pharmaceutical interest, for instance) is administered alone, or each of initial BO-11X formulation or the additional component are administered separately.

### III. Adoptive Cell Therapy

Adoptive Cell Therapy is a form of passive immunization by the transfusion (adoptive cell transfer) of immune cells, in particular T-cells. T cells are found in blood and tissue and usually activate when they find foreign pathogens or other antigens that T-cell's surface receptors encounter parts of foreign proteins (antigens) that are displayed on surface of other cells. These latter cells can be either infected cells, or antigen presenting cells (APCs) that are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death.

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the expansion and the reinfusion of the resulting cells. Activation can take place through gene therapy, or by exposing the T cells to tumor antigens. Additional details on the preparation, selection, use, combination with other therapies, an/or administration of cells for ACT treatment are described in the literature (Cook K et al., 2018, Elahi R et al., 2018; Sharma P. et al., 2017).

In some embodiments, the adoptive cell therapy comprises dendritic cell therapy, which provokes anti-tumor responses by causing dendritic cells to present tumor antigens to lymphocytes, and then activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T. One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF. Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies may include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

In some embodiments, the adoptive cell therapy comprises CAR-T cell therapy. Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy. Exemplary CAR-T therapies include Tisagenlecleucel (Kymriah) and Axicabtagene ciloleucel. In some embodiments, the CAR-T therapy targets CD19 or CD20.

### IV. Additional Therapies

The current methods and compositions of the disclosure may include one or more additional therapies known in the art and/or described herein. In some embodiments, the additional therapy comprises an additional cancer treatment. Examples of such treatments are described herein.

In some embodiments, the additional therapy comprises an oncolytic virus. An oncolytic virus is a virus that preferentially infects and kills cancer cells. In some embodiments, the additional therapy comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants. In some embodiments, the additional therapy comprises neoantigen administration. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden.

In some embodiments, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent. Suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

In some embodiments, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some embodiments, the additional therapy or prior therapy comprises surgery Upon excision of part or all of cancerous cells, tissue, or tumor, a cavity may be formed in the body. Treatment may be accomplished by perfusion, direct injection, or local application of the area with an additional anti-cancer therapy. Such treatment may be repeated. Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

In some embodiments, the methods comprise or exclude administration of a cancer immunotherapy. Cancer immunotherapy (sometimes called immuno-oncology, abbreviated 10) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Embodiments of the disclosure may include administration of ICB therapies, which are further described below.

In some embodiments, the immunotherapy comprises an inhibitor of a co-stimulatory molecule. In some embodiments, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

In some embodiments, the immunotherapy comprises cytokine therapy. Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins. Interferons are produced by the immune system. They are usually involved in anti-viral response, but also have use for cancer. They fall in three groups: type I (in particular IFNalpha and IFNbeta), type II and type III. Interleukins have an array of immune system effects. IL-2 is an exemplary interleukin cytokine therapy.

### V. Immunotherapy

In some embodiments, the methods comprise or exclude administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines.

Embodiments of the disclosure may include administration of ICB therapies, which are further described below.

A. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

B. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference. A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WOO 1/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein.

### VI. Sample Preparation

In certain aspects, methods involve obtaining a biological sample from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In certain embodiments the sample is obtained from a biopsy from esophageal tissue by any of the biopsy methods previously mentioned. In other embodiments the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A biological sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The biological sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as multiple esophageal samples may be obtained for diagnosis by the methods described herein. In other cases, multiple samples, such as one or more samples from one tissue type (for example esophagus) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

### VII. Methods of Treatment

Provided herein are methods for treating or delaying progression of cancer in an individual. In some embodiments, the individual has cancer that is resistant (has been demonstrated to be resistant) to one or more anti-cancer therapies. In some embodiments, resistance to anti-cancer therapy includes recurrence of cancer or refractory cancer. Recurrence may refer to the reappearance of cancer, in the original site or a new site, after treatment. In some embodiments, resistance to anti-cancer therapy includes progression of the cancer during treatment with the anti-cancer therapy. In some embodiments, the cancer is at early stage or at late stage.

The cancer may specifically be defined according to its histological type, but preferably the cancer may be a solid tumor, either a metastatic cancer or a non-metastatic cancer. In certain embodiments, the cancer may originate in the bladder, blood, bone, bone marrow, brain, breast, urinary, cervix, esophagus, duodenum, small intestine, large intestine, colon, colorectal, rectum, anus, gum, head, kidney, liver, lung, nasopharynx, neck, ovary, prostate, skin, stomach, testis, tongue, thymus or uterus.

The cancer may specifically be of the following type, though it is not limited to these, but preferably being solid and/or injectable cancer: cutaneous squamous-cell, noncolorectal gastrointestinal, colorectal, melanoma, Merkel-cell, renal-cell, cervical, hepatocellular, urothelial, non-small cell lung, head and neck, endometrial, esophagogastric, small-cell lung mesothelioma, ovarian, esophogogastric, glioblastoma, adrencorical, uveal, pancreatic, germ-cell, giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; cutaneous melanoma, blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant.

In some embodiments, the cancer comprises cutaneous squamous-cell carcinoma, non-colorectal and colorectal gastrointestinal cancer, Merkel-cell carcinoma, anal cancer, cervical cancer, hepatocellular cancer, urothelial cancer, melanoma, lung cancer, non-small cell lung cancer, small cell lung cancer, head and neck cancer, kidney cancer, bladder cancer, Hodgkin's lymphoma, pancreatic cancer, or skin cancer.

In some embodiments, the cancer comprises lung cancer, pancreatic cancer, metastatic melanoma, kidney cancer, bladder cancer, head and neck cancer, or Hodgkin's lymphoma.

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

In certain aspects, further cancer or metastasis examination or screening, or further diagnosis such as contrast enhanced computed tomography (CT), positron emission tomography-CT (PET-CT), and magnetic resonance imaging (MRI) may be performed for the detection of cancer or cancer metastasis in patients determined to have a certain gut microbiome composition.

### VIII. Administration of Therapeutic Compositions

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined quantity of the therapeutic composition. The quantity to be administered, and the particular route and formulation, is within the skill of determination of those in the clinical arts, in particular for intratumoral injection. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 0.1 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels, where applicable), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### IX. Kits

Certain aspects of the present disclosure also concern kits containing compositions of the disclosure or compositions to implement methods of the disclosure. In some embodiments, kits can be used to evaluate one or more biomarkers, for instance those identified among the genes that is upregulated by the administration of nanoplexed formulation of a TLR3 agonist such as BO-112 (see FIG. 10) or genetic mutations in the interferon receptor signaling pathway that result in innate resistance to PD-1 blockade immunotherapy (see US20180051347). The kits may include probes, primers, primer sets, synthetic molecules, binding reagents, PCR reagents, etc. for detecting the biomarkers described herein in a subject. In some embodiments, there are kits for evaluating biomarker activity in a cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, probes, and inhibitors are included in some kit embodiments. In addition, a kit may include a sample that is a negative or positive control for one or more biomarkers.

It is contemplated that any method or composition described herein can be implemented with respect to any other method or composition described herein and that different embodiments may be combined. The claims originally filed are contemplated to cover claims that are multiply dependent on any filed claim or combination of filed claims.

Embodiments of the disclosure include kits for analysis of a pathological sample by assessing biomarker profile for a sample comprising, in suitable container means, two or more biomarker probes, wherein the biomarker probes detect one or more of the biomarkers identified herein. The kit can further comprise reagents for labeling nucleic acids in the sample. The kit may also include labeling reagents, including at least one of amine-modified nucleotide, poly(A) polymerase, and poly(A) polymerase buffer. Labeling reagents can include an amine-reactive dye.

### EXAMPLES

### Example 1: Effects of Adoptive Cell Therapy Formulations in Tumor-Specific T Cells in B16 Murine Melanoma Models with Distinct Disruptions in Immune Pathways

The impact of tumor-intrinsic defects in interferon signaling on the efficacy of adoptively transferred tumor-specific T cells is unclear. We examined how type I and/or II interferon signaling defects in tumor cells impact the efficacy of adoptively transferred T cells. Although defects in either type I or II interferon signaling result in resistance to immune checkpoint blockade, such defects did not impact the efficacy of adoptively transferred T cells. Only defects in both type I and II tumor interferon signaling disrupt the efficacy of adoptively transferred T cells. This is a direct result of the dependency of MHC class I expression on type I or II interferon signaling.

Defects in interferon signaling have been described as a mechanism of resistance to cancer immunotherapy, but the mechanism by which these defects prevent T cell mediated anti-tumor efficacy is less clear. US20180051347 has described how specific mutations in the Interferon Signaling Pathway and antigen presentation pathway lead to either acquired or primary resistance to anti-PD-1 therapy.

To investigate the impact of tumor interferon signaling on anti-tumor efficacy of T cells we examined the impact of type I and/or II interferon signaling defects on the efficacy of adoptive cell therapy (ACT) with tumor-specific T cells in C57BL/6 mice using B16 murine melanoma model. At this scope, three distinct B16 murine melanoma cell lines were produced using CRISPR, each cell line having a gene involved in interferon signaling being disrupted as described in US20180051347: B16 IFNAR1^{KO} (deficient in type I interferon signaling), B16 JAK2^{KO} (deficient in type II interferon signaling), and B16 JAK1^{KO} (deficient in both type I and type II interferon signaling).

This evaluation of ACT effects was performed using gp100-specific pmel T cells, with BL/6 T cells as control, together with Interleukin-2 administration. Adoptive transfer of 4×10⁶ activated T cells on day 5 was performed in C57BL/6 mice pretreated with 5 Gy total body irradiation. BL/6 T cells were activated with anti-CD3/CD28 and IL2 and Pmel T cells were activated with gp100 peptide and IL2, (IL2 was administered i.p. on days 5-7).

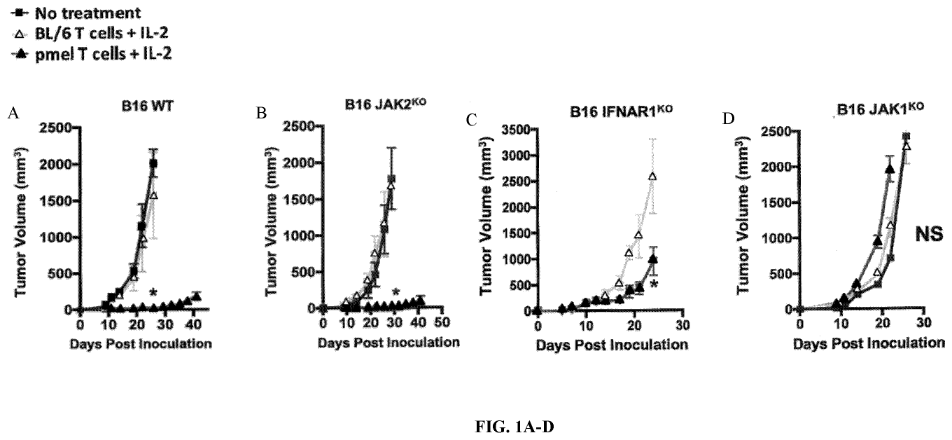

Pmel-based ACT was effective against B16 tumors lacking type I (B16 IFNAR1^{KO}) or type II (B16 JAK2^{KO}) interferon signaling, but ineffective against B16 tumors lacking both signaling pathways (B16 JAK1^{KO}) where ACT effects over tumor growth were not statistically significative (FIG. 1). A similar phenomenon was observed in vitro, where growth of B16 tumor cells lacking type I or II interferon signaling (but not both) were inhibited by tumor-specific pmel T cells compared to non-specific T cells, provided the alternate interferon pathway was activated (p<0.0001).

The interferon-dependence of MHC-I expression was evaluated in vivo, observing that basal MHC-I expression in B16-based models is dependent on either type I or II interferon signaling. In vivo, tumors lacking type II interferon (B16 JAK2^{KO}) or type I interferon (B16-IFNAR1-KO) signaling were able to augment MHC-I expression compared to a further B16 model in which B2M gene was disrupted (B16 B2M^{KO}; p=0.068). B16 JAK1^{KO} tumors did not express MHC-I in vivo, similar to B16-B2M-KO tumors.

### Example 2: Effects of Adoptive Cell Therapy and Nanoplexed Poly(I:C) Formulations in Tumor-Specific T Cells in B16 Murine Melanoma Models with Distinct Disruptions in Immune Pathways

We used genetic and pharmacologic approaches to study the impact of tumor-intrinsic interferon signaling on the direct anti-tumor efficacy of tumor-specific T cells. We performed in vitro and adoptive transfer studies using tumor-specific T cells against a murine model of melanoma with type I, type II and both type I and II interferon signaling defects. Only defects in both type I and II tumor interferon signaling disrupted the efficacy of adoptively transferred tumor-specific T cells, a byproduct of the dependency of MHC class I expression on either type I or II interferon signaling.

We reasoned that pharmacologic activation of pattern recognition receptor pathways may activate downstream signaling pathways redundant with IFN signaling, and in so doing, restore the efficacy of tumor-specific T cells against tumors with deficient IFN signaling and insufficient MHC I expression. To test this hypothesis, We have evaluated an intratumoral approach using BO-112, nanoplexed formulation of poly I:C, to uncouple tumor IFN signaling and MHC class I antigen presentation through activation of double-stranded RNA (dsRNA) sensing and NF-kB signaling, thereby restoring the efficacy of tumor-specific T cells. In a phase 1 study, BO-112 was found to be safe as monotherapy or in combination with anti-PD1 immune checkpoint blockade in patients with solid tumors (Marquez Rodas I et al., 2018).

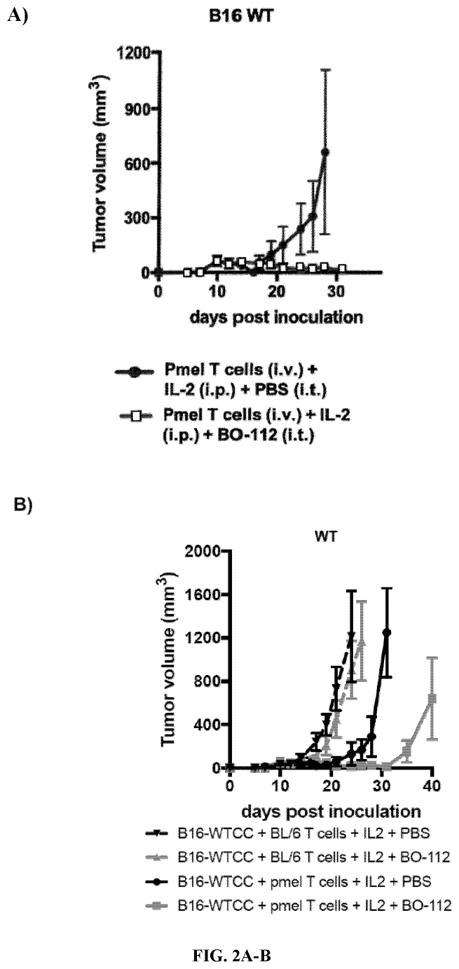

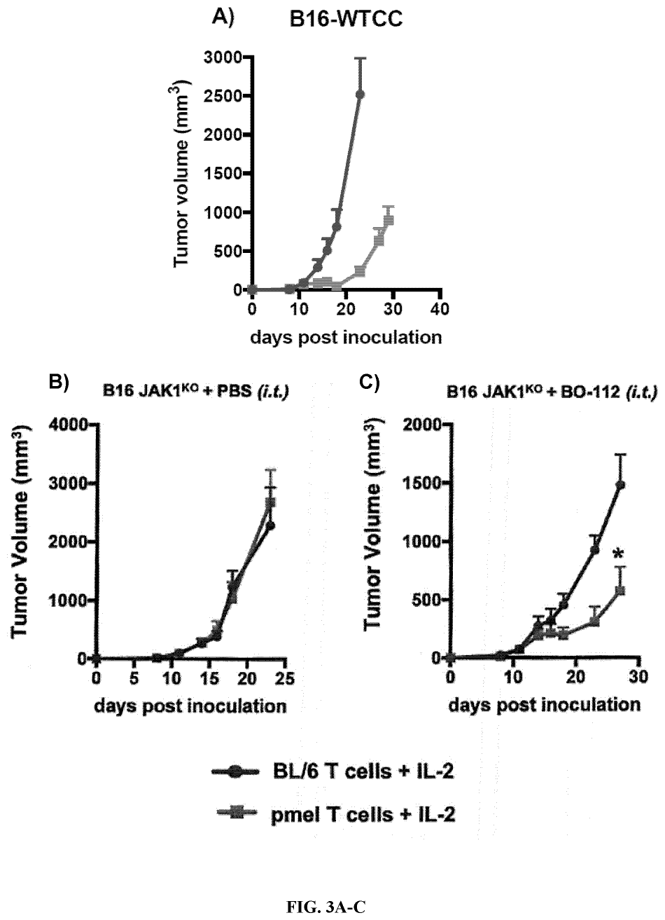

The B16-based tumor model was also tested using a nanoplexed formulation of poly(I:C) molecules that activates TLR3/MDA5/RIG-I signaling (BO-112) as a potential mechanism to improve anti-tumor activity of ACT, in general and with respect to type I/II interferon deficient tumors, as established in the models described above. Intratumoral delivery of BO-112, which has direct anti-tumor efficacy against B16, augments anti-tumor efficacy of pmel ACT against wildtype B16 (FIG. 2). The anti-tumor effects of BO-112 were further evaluated in the pmel ACT-resistant model of B16 JAK1^{KO} tumor cells. In combination with BO-112, pmel ACT was effective in C57BL/6 mice where B16 JAK1^{KO} tumors were raised, in particular when compared to non-specific T cells in combination with BO-112 (FIG. 3). Thus, the administration of intratumoral BO-112 not only improves efficacy of ACT in tumor that present a normal I and II interferon signaling but also resensitizes tumors lacking both type I and II interferon signaling (that is, not sensitive to ACT alone) to ACT, expanding the possibilities for using ACT for treating cancer in subjects presenting some type of defect or disruptions type I and II interferon signaling.

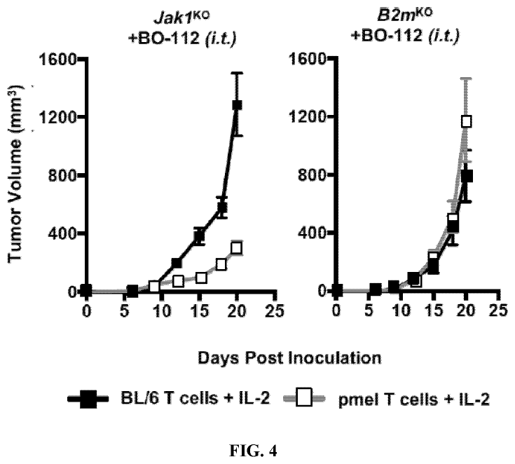

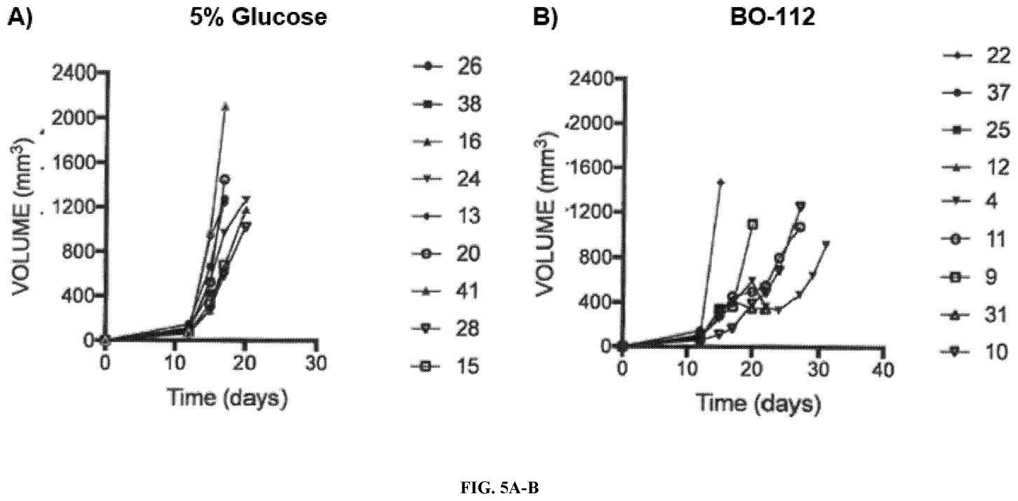

Moreover, like B16-Jak1^{KO} tumors, B16-B2m^{KO} tumors are also resistant to adoptively transferred pmel T cells (FIG. 4). However, unlike its effect on B16-Jak1^{KO} tumors, BO-112 did not restore the anti-tumor effect of adoptively transferred pmel T cells against B16-B2m^{KO} tumors. Of note, BO-112 also significantly augments the anti-tumor efficacy of adoptively transferred pmel T cells against wildtype B16 tumors, even though it is ineffective as monotherapy in the latter B2M model.

### Example 3: Effects of Adoptive Cell Therapy and Nanoplexed Poly(I:C) Formulations in Tumor-Specific T Cells in B16 Murine Melanoma Models with Distinct Disruptions in Interferon Signaling

Pharmacologically, BO-112, a potent nanoplex formulation of poly(I:C) that can be also delivered intratumorally, was observed to restore anti-tumor efficacy of adoptively transferred T cells against interferon-defective tumors. BO-112, by activating dsRNA sensing, may induce MHC class I expression through an NF-kB mediated pathway, independent of both interferon signaling and Nlrc5.

Additional experiments have been conducted to assess the direct anti-tumor effects of BO-112 in the B16 B2M^{KO}. BO-112 was able to significantly delay the growth of tumors compared to glucose treatment alone (FIG. 5). Synergistic effects in combination with ACT in this model are shown in this combination approach, suggesting that nanoplexed poly(I:C) formulations can also resensitize the tumors lacking a functional MHC-1 molecular machinery to ACT therapy. Thus, we demonstrate how the efficacy of adoptive T cell therapy tumors lacking interferon-signaling can be improved, insofar as MHC class I expression is intact. For tumors lacking MHC class I expression as a result of deficient tumor-interferon signaling, dsRNA activation via BO-112 affords an alternative approach to activate MHC class I expression and restore the efficacy of adoptive T cell therapy.

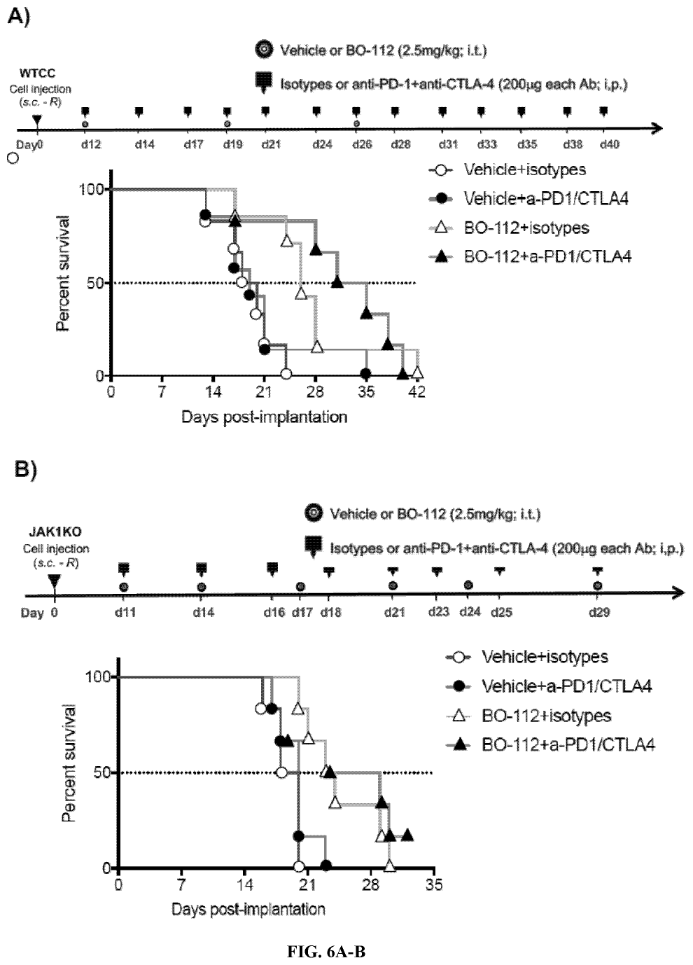

We also examined the effect of BO-112 on the efficacy of dual immune checkpoint inhibitor blockade (anti-CTLA4 and anti-PD1) against wildtype B16 and B16-Jak1^{KO} tumors, both of which are resistant to dual immune checkpoint blockade (FIG. 6). A consistent, but non-significant delay in tumor growth and prolonged survival was observed in wildtype B16 tumors treated with BO-112 plus dual checkpoint compared to BO-112 alone. However, no difference in tumor growth or survival was observed for B16-Jak1^{KO} tumors treated with BO-112 and dual checkpoint blockade compared to BO-112 alone. Thus, the addition of BO-112, even if providing a significant effect on survival when compared with vehicle-only treated animals, does not overcome resistance to dual immune checkpoint blockade in B16-Jak1^{KO} model.

### Example 4: In Vitro Evaluation of BO-112 Effects on the Expression of Cell Surface Proteins Using Standard and Genetically Modified Melanoma Cell Lines

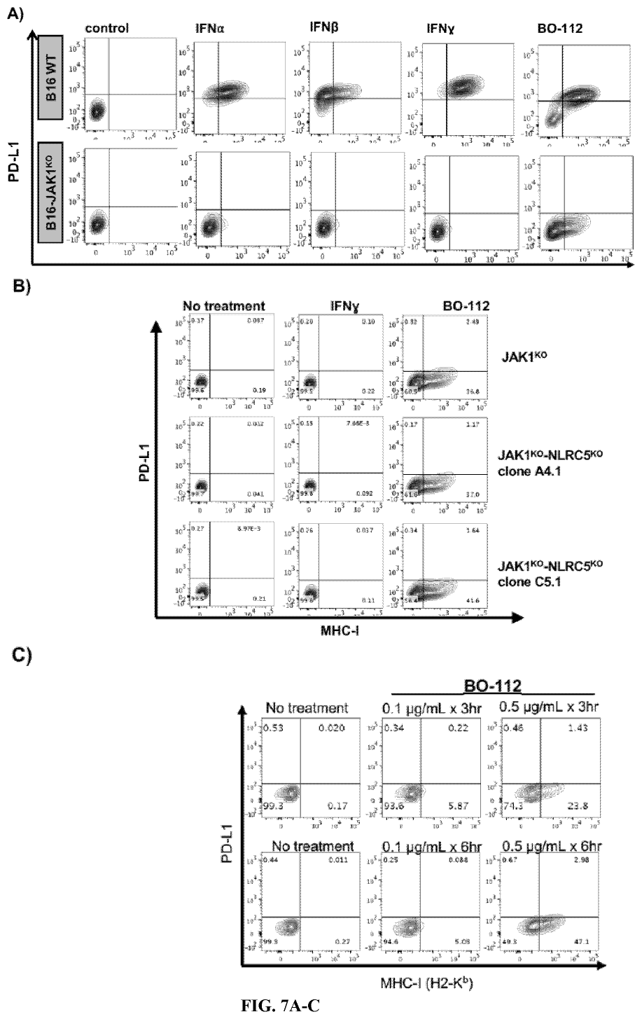

The BO-112 properties of therapeutic interest identified in the animal models have been further characterized by observing how in vitro exposure to BO-112 alters interferon-related gene expression in different genetic background, that is where one or more specific genes are inactivated. When tested by flow cytometry (FIG. 7), BO-112 clearly augments the expression of surface MHC I and PD-L1 expression of the wildtype B16 tumor cell line, similar to the effects of type I and II Interferons but, only BO-112 augments the expression of the B16-Jak1^{KO} and Jak1^{KO}-NLRC5^{KO} cell lines in a time and dose dependent manner.

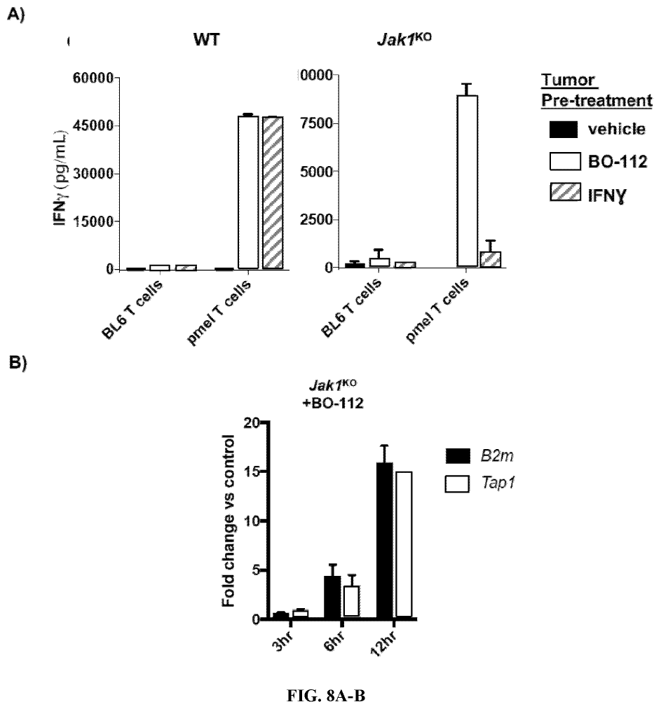

Tumor-specific IFNγ production by pmel T cells in coculture with B16 tumor cells occurs after pre-treatment of wildtype B16 tumor cells with either BO-112 or IFN gamma (IFNγ or IFNg). In contrast, pmel T cells only recognize B16-Jak1^{KO} tumor cells pre-treated with BO-112, but not IFNγ. Expression of MHC I antigen processing machinery genes B2m and Tap1 are augmented already within 6 hours of exposure to BO-112 (FIG. 8).

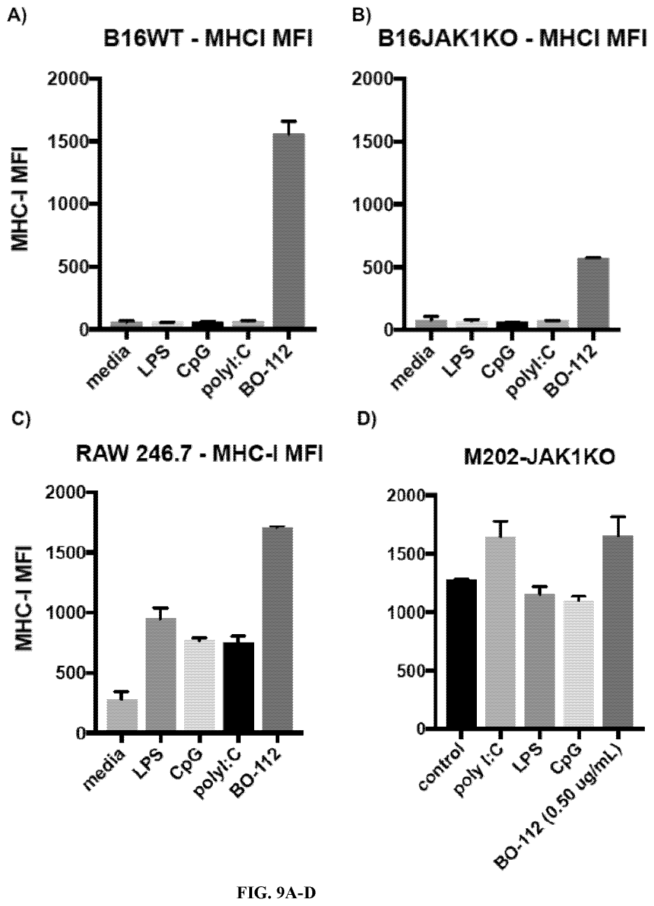

The putative molecular mechanism of BO-112 is through engagement of double stranded RNA (dsRNA) sensors, such TLR3, Rig-I, and/or MDA5. We compared the MHC I augmenting effects of BO-112 with the effects of a standard formulation of poly I:C, as well as two other pattern recognition receptor (PRR) agonists: lipopolysaccharide (LPS) and CpG oligonucleotides (FIG. 9). In a mouse macrophage cell line (RAW 246.7) known to respond to PRR agonists, LPS, CpG, poly I:C all resulted in an increase in MHC I expression, as did BO-112. However, aside from BO-112, none of the PRR agonists augmented MHC-I expression of the wildtype B16 or B16-Jak1^{KO} cell lines. The surface MHC I expression of the interferon-insensitive M202-JAK1^{KO} human melanoma cell line increased in response to both poly I:C and BO-112, but neither LPS or CpG, indicating an effect unique to dsRNA sensing.

In order to identify molecular mediators of the BO-112 activity, This effect has been evaluated at molecular level by RNA sequencing of tumors 5 days after ACT. This approach revealed more than 200 genes that are enriched (fold change >2, adjusted p-value<0.05) in tumors treated with pmel ACT and BO-112, which were not enriched in tumors treated with pmel ACT and vehicle or non-specific ACT and BO-112, including genes involved in T cell recruitment (Cxcl9, Ccl2, S1pr1), antigen presentation (Psmb8, Psmb9, Tap1), direct T cell cytotoxicity (Gzma, Gzmb, Prf1), and interferon signaling Ifng, Stat1, Mx1) RNA-sequencing analysis was performed in B16-Jak1^{KO} tumor cells six hours after treatment with vehicle or BO-112. A set of 795 genes differentially expressed in response to BO-112 (p<0.01, FDR<0.05, and Log₂(Fold Change)>1.5) was obtained and, after filtering for genes associated with differentially expressed gene sets and filtering gene sets to those with at least 30 genes differentially expressed, 195 genes and 12 pathways were identified (FIG. 10). Of note, despite the absence of IFN signaling in B16-Jak1^{KO} tumor cells, BO-112 induces an “IFN-like” gene signature, highlighted by genes in the KEGG JAK STAT Signaling Pathway, the Hallmark IFN Gamma Response Pathway, and the Hallmark IFN Alpha Response Pathway. To evaluate whether this in vitro effect of BO-112 on B16-Jak1^{KO} was relevant in vivo, 135 genes were identified as being upregulated in vivo by both BO-112 in combination with control T cells and pmel T cells. The relative expression of these 135 genes was sharply increased both in vitro and in vivo in groups treated with BO-112. This gene set was enriched for genes involved in type I IFN signaling and TNF-alpha signaling via NF-kB but other genes are also specifically induced, defining a gene expression profile that characterize the response in cancer cells to BO-112 exposure. The genes that are found over expressed by BO-112 administration, in particular those presenting at least 5.0 of Log 2 fold increase (each of them singularly or combined in set of 2, 3, 5, 10 or more of them) can be used as biomarker of the biological response to BO-112 and exploited to define improved means to use and adapted regimens for administering BO-112, with respect to Adoptive Cell Therapy or more in general.

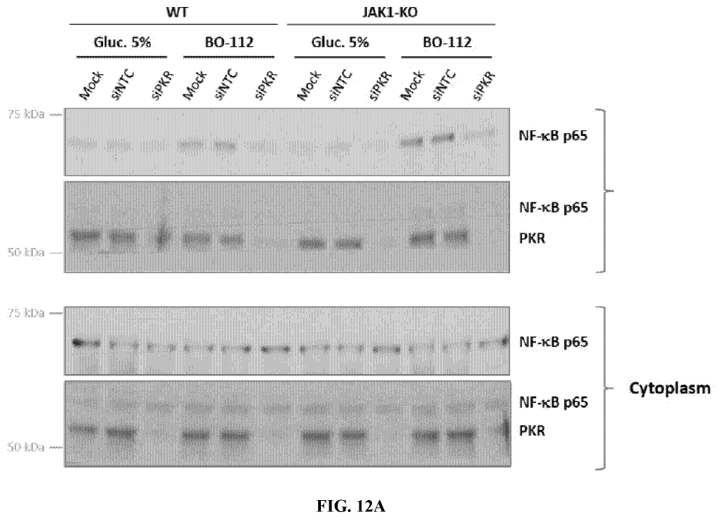

In order to evaluated whether NF-kB was the transcriptional effector of the signaling induced by BO-112 that results in IFN- and Nlrc5-independent MHC I expression. To do this, B16-Jak1^{KO} cell line was treated with BO-112 in conjunction with a selective NF-kB inhibitor, BMS-345541 (FIG. 11). BMS-345541 abrogates the induction of MHC I by BO-112 in a dose-dependent manner. A transient knockdown of Rela via two different siRNAs achieved a similar effect, inhibiting the upregulation of surface MHC I by BO-112 in B16-Jak1^{KO} tumors. Knockdown of RELA in the M202-JAK1^{KO} human melanoma cell lines also disrupts upregulation of MHC I by BO-112.

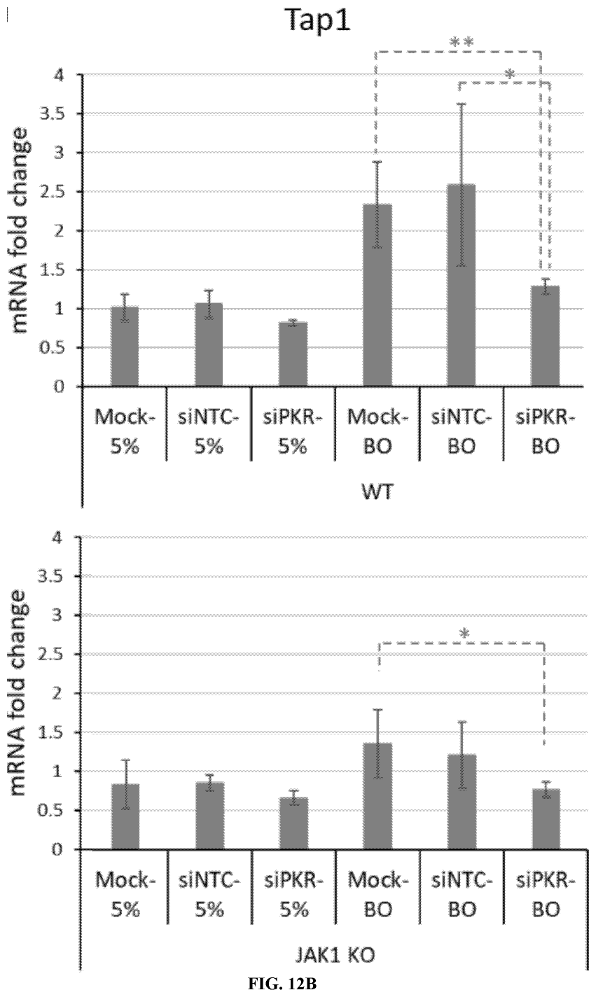

Among the dsRNA sensors, protein kinase RNA-activated (PKR) is known to signal downstream through NF-kB, in addition to other downstream signaling pathways. BO-112 induced nuclear translocation of NF-kB (p65) in both wildtype B16 and B16-Jak1^{KO} tumor cell lines (FIG. 12). This effect was mediated by PKR, as siRNA for PKR reduced the levels of nuclear NF-kB (p65) in response to BO-112. The siRNA targeting PKR also reduced induction of Tap1 gene expression in response to BO-112.

### Example 5: Materials & Methods

### A. Cell Lines

Human cell lines and B16-F10 cell lines were purchased from ATCC and cultured with complete media (RPMI 1640) containing 10% fetal bovine serum (FBS; Omega), penicillin (100 U/mL), streptomycin (100 μg/mL), and ampicillin. Cell lines were confirmed mycoplasma negative using mycoplasma detection kit (Biotool, Cat. no. B3903). Mice were bred and kept under defined-flora, pathogen-free conditions at the Association for the Assessment and Accreditation of Laboratory Care-approved animal facility of the Division of Experimental Radiation Oncology, University of California, Los Angeles (UCLA). Pmel-1 TCR/Thy1.1 transgenic mice on a C57BL/6 background were obtained from the Jackson Laboratory (Bar Harbor, Me., USA) and splenocytes were cultured in RPMI 1640 media supplemented with 10% FCS, antibiotics, 50 uM 2-mercaptoethanol (Gibco), murine IL-2 (Peptrotech), and murine gp100 peptide (Fisher). Thy1.1 C57BL/6 non-transgenic mice were used as a control, and splenocytes were cultured in RPMI 1640 media supplemented with 10% FCS, antibiotics, 50 μM 2-mercaptoethanol (Gibco), murine IL-2 (Peprotech), and pulsed with anti-CD3 and anti-CD28 antibodies.

### B. CRISPR/Cas9 Knockout of Interferon Pathway Genes

Gene targeting by CRISPR/Cas9 was accomplished by transfection of the guide sequence (selected using the CRISPOR program) cloned into the Cas9 plasmid (pSpCas9(BB)-2A-GFP, PX458; Addgene, cat no. 48138) containing an ampicillin selection marker as previously described (Ran et al., 2013). Along with the Cas9 encoded, the plasmid contains a gene block of 20 bp target size (N), U6 promoter, termination signal, and GFP. Lipofectamine 3000 reagent (Fisher) was used to transfect cell lines. Knockout cells were sorted from a bulk knockout population using Fluorescence Activated Cell Sorting (FACS) on the Aria II (BD). Successful targeting of genes of interest was determined by tracking of indels by decomposition (TIDE) analysis (Netherlands Cancer Institute NKI; https://tide.nki.ni), as well as treatment of cells with and without 100 ng/mL of interferon (IFN)-gamma (Peprotech), 5000 IU/mL of IFN-beta (PBL Assay Science), 500 IU/mL of IFN-alpha (PBL Assay Science), or 1.0 μg/mL of BO-112 (Bioncotech Therapeutics; WO2017085228) and examining PD-L1 and MHC-I surface expression by flow cytometry.

### C. Flow Cytometry Analysis

B16-F10 wild-type and knockout cell lines were seeded in complete media containing 100 ng/mL of IFN-gamma, 500 IU/mL of IFN-beta, 500 IU/mL of IFN-alpha, 1.0 μg/mL BO-112 (Bioncotech Therapeutics; WO2017085228), BMS-345541 (Sigma-Aldrich; cat. no. B9935), or PBS for 18 hours. Interferon concentrations were determined as previously described (Garcia-Diaz et al., 2017). After 18 hours, cells were harvested with 10 mM EDTA (Sigma-Aldrich) and surface-stained in phosphate-buffered saline (PBS), 5% fetal bovine serum, and 2 mM EDTA with allophycocyanin (APC) anti-MHC-I (Biolegend) and phycoerythrin (PE) PD-L1 (Biolegend) antibodies. Cells were analyzed by flow cytometry using a LSRII (BD Biosciences). Data was analyzed using the FlowJo software (Tree Star).

### D. Coculture Assays

B16-F10 wildtype and knockout cell lines (RFP+/RFP−) were pulse with 100 ng/mL of IFN-gamma and 500 IU/mL of IFN-beta 18 hours before coculture, and 0.5 ug/mL of BO-112 (Bioncotech Therapeutics) 6 hours before coculture. After 18 hours, RFP-positive murine melanoma cells were harvested using 10 mM EDTA and plated in a flat bottom 96-well plate in triplicate for each condition at 5000 cells per well for IncuCyte Live Cell Analysis (Essen Bioscience). After 18 hours, non-RFP murine melanoma cells were harvested using 10 mM EDTA and plated in a round bottom 96-well plate in triplicate for each condition at 100,000 cells per well for ELISA analysis. Following plating of the melanoma cell lines, pmel-1 T-cells and C57BL/6 splenocytes were harvested and plated in the flat bottom 96-well plate at 10,000 cells per well, and plated in the round bottom 96-well plate at 100,000 cells per well. The flat-bottom 96 well plate was then placed in the IncuCyte for 72 hours. ELISA analysis plate was incubated at 37° C. for 24 hours. Supernatant was then harvested and frozen at −20° C. for further analysis.

### E. ELISA

Coculture supernatants were analyzed by ELISA for mouse IFN-gamma (ThermoFisher) according to the manufacturer's instructions.

### F. Gene Expression Assays

Total RNAs were extracted using the PureLink RNA Mini Kit (Invitrogen) from B16 cell lines untreated and treated with BO-112. Gene expression was then measured using the Power SYBR Green RNA-to-CT 1-Step Kit (ThermoFisher) according to the manufacturer's instructions. RT-PCR was performed by using the ViiA 7 Real-Time PCR System (ThermoFisher).

### G. Murine NLRC5 Plasmid Design

Total RNA was obtained from murine splenocytes using the PureLink RNA Mini Kit (Invitrogen) according to the manufacturer's instructions. Total RNA was then reverse transcribed to cDNA using the Superscript IV Reverse Transcriptase Kit (ThermoFisher) with an Oligo(dT)20 primer (ThermoFisher). The cDNA was then amplified using the Phusion High-Fidelity PCR Kit (New England BioLabs). Following cDNA amplification, a Gibson Assembly (New England BioLabs) was used to incorporate the new cDNA into the pRRL-MSCV viral plasmid.

### H. Lentiviral Vector Production and Gene Transfer

Lentivirus production was achieved by co-transfection of 293T cells (ATCC). 10 cm cell culture dishes, coated with poly-L-lysine (Sigma Aldrich) containing 5×10⁶ 293T cells were used for transfection. The constructs for the lentivirus—pRRL-MSCV-mNLRC5 and pRRL-MSCV-mGFP (5 ug), pCMV8.9 (5 ug), and pCAGGS-VSV-G (1 μg) were added to water for a total volume of 50 μL. TransIT-293 Transfection Reagent (MirusBio) was used in conjunction with the diluted DNA mixture, and the complex was added dropwise to the 10 cm dishes. After 17 hours, the media was aspirated and replaced with DMEM medium with 10% fetal bovine serum containing 20 mM HEPES (Invitrogen) and 10 mM Sodium Butyrate (Sigma Aldrich). After 8 hours, cells were washed and fresh complete DMEM medium with 20 mM HEPES was added. After 24 hours, the supernatants were collected, filtered through 0.45 uM filters, and stored in −80° C. For transduction, 5×10⁵ cells were plated in 6 well plates overnight. Virus supernatant was added to each well, along with 8 ug/mL of polybrene (Sigma Aldrich). After 12-16 hours, media was replaced.

### I. In Vivo Inoculations and Animal Studies

B16-F10 tumor cells were injected subcutaneously on both the right and left sides of the abdomen of C57BL/6 mice. 7 days after tumor inoculation, mice were treated with lymphoid depleting (500 cGy) total body irradiation. On day 9, mice received gp100-activated pmel-1 splenocytes intravenously and received intraperitoneal injections of 50,000 IU of human interleukin 2 (IL-2) for 3 days. Beginning day 9, BO-112 was administered via intratumoral injection at 2.5 mg/kg, twice a week. Activated splenocytes from wild-type C57BL/6 mice were used as controls. Tumor size was monitored on both sides every two to three days and expressed as volume (mm³).

### J. Statistical Analysis

Prism software (GraphPad) was utilized to analyze tumor growth and determine statistical significance between treated and untreated groups by using the unpaired Student's t test, with p-values<0.05 determined significant.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### X. References

The following references, to the extent that they provide exemplary procedural or other details supplementary to those set forth herein, are specifically incorporated herein by reference.
Bousoik E and Montazeri Aliabadi H, Front Oncol. 2018; 8:287.
Cook K et al., Biomedicines. 2018; 6(2): 37.
Elahi R et al., Front Immunol. 2018; 9:1717.
Galluzzi L et al., Sci Transl Med. 2018; 10(459). pii: eaat7807.
Kadam, R N et al., Braz. J. Pharm. Sci. 2015; 51(2): 255-263.
Marquez Rodas I et al., Annals of Oncology 2018; 29 (suppl_8), mdy424.049,
Patel M C et al., Future Virol. 2014 September; 9(9):811-829.
Sharma P, et al., Cell. 2017; 168(4):707-723.
Tormo D et al., Cancer Cell. 2009; 16(2):103-14.
Garcia-Diaz A et al., Cell Rep. 2017 9; 19(6):1189-1201.
Ran et al Nat Protoc. 2013 November; 8(11):2281-308.
US20180051347
WO2017085228
WO2018210439

1. A method of treating a subject having cancer, comprising administering an Adoptive Cell Therapy in combination with a nanoplexed formulation of a TLR3 agonist wherein said nanoplexed formulation of a TLR3 agonist comprises a complex formed by polyinosinic-polycytidylic acid [poly(I:C)] molecules and linear polyethyleneimine.

2. The method of claim 1, wherein the nanoplexed formulation of a TLR3 agonist is administered by intratumoral injection.

3. The method of claim 1 wherein said nanoplexed formulation is administered to the subject at the time of or after Adoptive Cell Therapy.

4. The method of claim 1, wherein the nanoplexed formulation of the TLR3 agonist and the Adoptive Cell Therapy are administered within 1 day of each other.

5. The method of claim 1, wherein the Adoptive Cell Therapy comprises the administration of tumor-infiltrating lymphocytes, in vitro and/or ex vivo modified or sensitized immune cells, chimeric antigen receptor (CAR) cell therapy, and/or engineered T cell receptor (TCR) cell therapy.

6. The method of claim 1, wherein the method further comprises administration of an additional therapy or wherein the subject has previously received an additional therapy or will receive an additional therapy.

7. The method of claim 6, wherein the subject has been determined to be a non-responder to the additional therapy.

8. The method of claim 6, wherein the subject has been determined to have a toxic response to the additional therapy.

9. The method of claim 6, wherein the additional therapy comprises a cytokine therapy.

10. The method of claim 6, wherein the additional therapy comprises an immunotherapy.

11. The method of claim 10, wherein the immunotherapy comprises immune checkpoint blockade (ICB) therapy.

12. The method of claim 10, wherein the ICB therapy comprises one or more of anti-PD-1 therapy, an anti-PD-L1 therapy, or an anti-CTLA-4 therapy.

13-21. (canceled)

22. The method of claim 1, wherein the cancer is a solid cancer.

23. The method of claim 22, wherein the cancer is an injectable cancer, or a cancer that that can be treated by intratumoral injection.

24. The method of claim 23, wherein the cancer is skin cancer, non-small cell lung cancer, endometrial cancer, kidney cancer, bladder cancer, colon or colorectal cancer, breast cancer, prostate cancer, lung cancer, head and neck cancer, pancreatic cancer, genitourinary cancer, ovarian cancer, rectal cancer, gastric cancer, sarcoma, and esophageal cancer.

25. The method of claim 1, wherein the cancer comprises a recurrent cancer.

26. The method of claim 1, wherein the cancer is unresponsive or refractory to other therapies.
